(19) United States
(12) Patent Application Publication
(10) Pub. No.: US 2026/0335472 A1
(43) Pub. Date: Oct. 29, 2026

(54) MULTIFUNCTIONAL COMPOUND CORE FOR ACTIVE AGENTS AND USES THEREOF

(71) Applicant: THE UNIVERSITY OF NORTH CAROLINA AT CHAPEL HILL, Chapel Hill, NC (US)

(72) Inventor: Zibo Li, Chapel Hill, NC (US)

(21) Appl. No.: 19/481,062

(22) PCT Filed: May 3, 2024
(86) PCT No.: PCT/US2024/027706

§ 371 Date: Nov. 3, 2025

Related U.S. Application Data
(60) Provisional application No. 63/499,880, filed on May 3, 2023.

(51) Int. Cl. A61K 51/04; C07D 251/32; C07D 403/12; C07D 515/08; C07D 515/22

(52) U.S. Cl. CPC A61K 51/0497; A61K 51/0461; C07D 251/32; C07D 403/12; C07D 515/08; C07D 515/22

(57) ABSTRACT

Provided are multifunctional compound cores that can be functionalized with specific R groups to act as theranostic agents, i.e., for the imaging and/or treating of a cancer in a subject. Methods of synthesizing said compounds and methods of treating a subject by administering a compound that is described herein are also provided.

### CROSS-REFERENCE TO RELATED APPLICATIONS

This application claims the benefit of U.S. Provisional Application Ser. No. 63/499,880, filed May 3, 2023, the contents of which are incorporated by reference herein.

### FIELD OF THE INVENTION

This invention relates to multifunctional compound cores for delivery of active agents (e.g., theranostic agents) to a subject, methods of using said compounds for imaging and/or treating a disease or disorder (e.g., cancer) in a subject, and methods of synthesizing said compounds.

### BACKGROUND OF THE INVENTION

The development of theranostic agents based on radionuclides, which are capable of serving as both diagnostic and therapeutic agents in one compound, have been a major focus for cancer therapy research in the past few years. In fact, major cancer centers throughout the world have established radionuclide-based theranostic programs in the past few years. One beneficial feature of radionuclide therapy is that the compound does not need to block the signaling pathway of the receptors in order to be an effective treatment. The radiation from the bound radionuclide is often able kill the cancer cells. In addition, a radionuclide therapy can be easily combined with immunotherapy, chemotherapy, and gene therapy in order to boost the treatment efficacy. Moreover, the use of specific targeting ligands for receptors that are highly expressed in cancer cells allows a high tumor to background contrast ratio. The same radioligands used for therapies can also be labeled with a PET isotope for patient screening, greatly facilitating personalized medicine as only patients with a high uptake in PET scans will proceed with radionuclide therapies. Novel compounds for delivery of diagnostic, therapeutic, and theranostic agents are desirable to improve patient management in many different diseases and disorders.

### SUMMARY OF THE INVENTION

One aspect of the invention provides a compound comprising a compound core, wherein the compound core is covalently linked to at least one of a targeting ligand, a chelating agent, an imaging agent, a therapeutic agent, an open reactive group, a radionuclide, a cyclic peptide, or any combination thereof; or a pharmaceutically acceptable salt thereof. In some embodiments, the compound core is a multifunctional (e.g., a bifunctional, trifunctional, or tetrafunctional) heterocycle, e.g., cyanuric acid, trithiocyanuric acid, triazine, barbituric acid, pyrimidine, Sar cage, 1,4,7,10-tetraazacyclododacane-1,4,7,10-tetraacetic acid (DOTA), 1,10-diaza-18-crown-6, alloxan, or a homodimer, heterodimer, homotrimer, heterotrimer, homotetramer, or heterotetramer thereof. In some embodiments, the compound core is covalently linked to at least two or to at least three of the targeting ligand, chelating agent, imaging agent, therapeutic agent, open reactive group, radionuclide, cyclic peptide, or any combination thereof.

Another aspect of the invention provides a method of synthesizing a compound, said method comprising attaching a targeting ligand, chelating agent, imaging agent, therapeutic agent, peptide reactive agent, and/or cyclic peptide to a compound core using, e.g., alkylation, amide bond formation, reductive amination, nucleophilic substitution reaction, click chemistry, tetrazine-trans-cyclooctene (TTCO) ligation, urea bond formation, thiourea bond formation, phosphore bond formation, epoxide ring opening, and/or a reaction with thianthrenium salt (TT).

Another aspect of the invention provides a method of detecting a tumor in a subject in need thereof, comprising administering to the subject a compound described herein and detecting binding of the compound to the tumor, wherein said compound comprises at least one imaging agent, at least one chelating agent or radionuclide, and at least one targeting ligand that targets the tumor.

Another aspect of the invention provides a method of treating cancer in a subject in need thereof, comprising administering to the subject a therapeutically effective amount of a compound described herein, thereby treating the cancer, wherein said compound comprises at least one therapeutic agent, at least one chelating agent or radionuclide, and at least one targeting ligand that targets the tumor.

Another aspect of the invention provides a method of detecting and treating a tumor in a subject in need thereof, comprising: a first step of administering a compound described herein to the subject and detecting the imaging agent, thereby determining the location and size of tumor; and a second step of administering the compound described herein to said subject wherein said compound is provided in a therapeutically effective amount; and wherein said compound comprises at least one therapeutic agent, at least one imaging agent, at least one chelating agent or radionuclide, and at least one targeting ligand that targets the tumor.

Another aspect of the invention provides a use of a compound described herein for detecting and treating a tumor in a subject in need thereof, or for the preparation of a medicament for detecting and treating a tumor in a subject in need thereof.

These and other aspects of the invention are set forth in more detail in the description of the invention below.

### BRIEF DESCRIPTION OF THE DRAWINGS

FIG. 1, panel A shows a PET scan image of a mouse showing that ⁶⁴Cu-NOTA-UNC-PSMA2 demonstrated high and persistent tumor uptake in a PC3-PSMA model. FIG. 1, panel B shows a PET scan image of a non-human primate showing that the salivary glands (SG) uptake of ⁶⁴Cu-NOTA-UNC-PSMA2 is only 1/10^{th} of ⁶⁴Cu-PSMA-617 at 24 hours post injection.

FIG. 2, panel A shows a PET scan image of a mouse showing that ⁶⁸Ga-PSMA-617 demonstrated clean tumor uptake profile in rodent models. FIG. 2, panel B shows a SPECT/CT scan image in a human subject showing that ¹⁷⁷Lu-PSMA-617 demonstrated high glands uptake at 24 hours post injection.

FIG. 3 shows a PET scan image of a mouse showing that two targeting ligands and a chelating agent attached to a single trifunctional compound core allows binding both to fibroblast activation protein (FAP) expressing and prostate-specific membrane antigen (PSMA) expressing tumors. The chelating agent is used to bind a radionuclide for imaging.

FIG. 4 shows a PET scan image of a mouse showing the uptake of a trifunctional core compound covalently linked to a targeting ligand, chelating agent, and therapeutic agent into tumors at 1 h, 4 h, 24 h, and 48 h.

FIG. 5 shows a PET scan image of a mouse showing the uptake of a bifunctional core compound covalently linked to a targeting ligand and imaging agent into a tumor.

FIG. 6 shows a PET scan images of mice showing the uptake of a PEG core compound covalently linked to a chelating agent and two targeting agents into a tumor at 1 hr and 4 h.

### DETAILED DESCRIPTION OF EMBODIMENTS OF THE INVENTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

The singular forms “a” and “an” are intended to include the plural forms as well, unless the context clearly indicates otherwise.

Furthermore, the term “about.” as used herein when referring to a measurable value such as an amount of the length of a polynucleotide or polypeptide sequence, dose, time, temperature, and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

Also as used herein, “and/or” refers to and encompasses any and all possible combinations of one or more of the associated listed items, as well as the lack of combinations when interpreted in the alternative (“or”).

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The terms “treat” or “treating” or “treatment” refer to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, etc., as would be well known in the art.

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. The effective amount may vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. See, for example, Remington. The Science and Practice of Pharmacy (20th ed. 2000).

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, sterile pyrogen-free water and sterile pyrogen-free physiological saline solution.

The term “administering” or “administration” of a composition of the present invention to a subject includes any route of introducing or delivering to a subject a compound to perform its intended function (e.g., for use in PET imaging, e.g., for the guidance of surgery).

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster, etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human.

A “subject in need” of the methods of the invention can be any subject known or suspected to have cancer and/or an illness to which imaging and/or surgery may provide beneficial health effects, or a subject having an increased risk of developing the same.

A “sample”, “biological sample”, and/or “ex vivo sample” of this invention can be any biological material, such as a biological fluid, an extract from a cell, an extracellular matrix isolated from a cell, a cell (in solution or bound to a solid support), a tissue, a tissue homogenate, and the like as are well known in the art.

As used herein, by “isolate” or “purify” (or grammatical equivalents) a compound and/or molecule, it is meant that the compound and/or molecule is at least partially separated from at least some of the other components in the starting material.

The terms “amino acid sequence,” “polypeptide.” “peptide” and “protein” may be used interchangeably to refer to polymers of amino acids of any length. The terms “nucleic acid,” “nucleic acid sequence,” and “polynucleotide” may be used interchangeably to refer to polymers of nucleotides of any length. As used herein, the terms “nucleotide sequence,” “polynucleotide,” “nucleic acid sequence,” “nucleic acid molecule” and “nucleic acid fragment” may refer to a polymer of RNA, DNA, or RNA and DNA that is single- or double-stranded, optionally containing synthetic, non-natural and/or altered nucleotide bases.

An “acyl” is intended to mean a group —C(O)—R, where R is a suitable substituent, such as alkyl, cycloalkyl, heterocycle, aryl, or heteroaryl. Examples of acyl include, but are not limited to, an acetyl group, a propionyl group, a butyroyl group, a benzoyl group (“Bz”), etc.

“Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11. C12, C13, C14, C15, etc.). In some embodiments the alkyl can be a lower alkyl. “Lower alkyl” refers to a straight or branched chain alkyl having from 1 to 3, or from 1 to 5, or from 1 to 8 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. In some embodiments, alkyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy. Representative examples of halo substituted alkyls include, but are not limited to, fluoromethyl, difluoromethyl and trifluoromethyl.

As generally understood by those of skill in the art, “saturation” refers to the state in which all available valence bonds of an atom (e.g., carbon) are attached to other atoms. Similarly, “unsaturation” refers to the state in which not all the available valence bonds are attached to other atoms; in such compounds the extra bonds usually take the form of double or triple bonds (usually with carbon). For example, a carbon chain is “saturated” when there are no double or triple bonds present along the chain or directly connected to the chain (e.g., a carbonyl), and is “unsaturated” when at least one double or triple bond is present along the chain or directly connected to the chain (e.g., a carbonyl). Further, the presence or absence of a substituent depending upon chain saturation will be understood by those of skill in the art to depend upon the valence requirement of the atom or atoms to which the substituent binds (e.g., carbon).

The term “optionally substituted” indicates that the specified group is either unsubstituted or substituted by one or more suitable substituents. A “substituent” that is “substituted” is an atom or group which takes the place of a hydrogen atom on the parent chain or cycle of an organic molecule, for example, halo, hydroxy, acyl, alkyl, alkenyl, alkynyl, cycloalkyl, heterocyclo, aryl, heteroaryl, alkoxy, amino, amide, thiol, sulfide, sulfone, sulfoxide, carbonyl, or carboxy.

“Alkenyl,” as used herein, refers to a straight or branched chain hydrocarbon containing from 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. In some embodiments, alkenyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Alkynyl,” as used herein, refers to a straight or branched chain hydrocarbon group containing from 2 to 10 or 20 or more carbon atoms, and containing at least one carbon-carbon triple bond. Representative examples of alkynyl include, but are not limited, to acetylenyl, 1-propynyl, 2-propynyl, 3-butynyl, 2-pentynyl, 1-butynyl and the like. In some embodiments, alkynyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

The term “cycloalkyl,” as used herein, refers to a saturated cyclic hydrocarbon group containing from 3 to 8 carbons or more. Representative examples of cycloalkyl include, cyclopropyl, cyclobutyl, cyclopentyl, cyclohexyl, cycloheptyl, and cyclooctyl. In some embodiments, cycloalkyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy. A representative example of a substituted cycloalkyl include epoxide.

“Heterocycle,” as used herein, refers to a monocyclic, bicyclic, or tricyclic ring system comprising at least one heteroatom. Monocyclic heterocycle ring systems are exemplified by any 4-, 5-, 6- or 7-member ring containing 1, 2, 3, or 4 heteroatoms independently selected from the group consisting of: 0, N, and S. The 4-member ring has 0 to 1 double bond, the 5-member ring has from 0 to 2 double bonds, and the 6 and 7 member rings have from 0 to 3 double bonds. Representative examples of monocyclic ring systems include, but are not limited to, azetidine, azepine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, thienothiophene and the like. In some embodiments, heterocyclo groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Aryl” as used herein refers to a ring system having one or more aromatic rings. Representative examples of aryl include azulenyl, indanyl, indenyl, naphthyl, phenyl, tetrahydronaphthyl, and the like. The aryl groups of this invention can be substituted with 1, 2, 3, 4, or 5 substituents independently selected from alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, aryloxy, azido, arylalkoxy, arylalkyl, aryloxy, carboxy, cyano, formyl, halogen, haloalkyl, haloalkoxy, hydroxy, hydroxyalkyl, mercapto, nitro, sulfamyl, sulfo, sulfonate. —NR′R″ (wherein, R′ and R″ are independently selected from hydrogen, alkyl, alkylcarbonyl, aryl, arylalkyl and formyl), and —C(O)NR′R″ (wherein R′ and R″ are independently selected from hydrogen, alkyl, alkylcarbonyl, aryl, arylalkyl, and formyl). In some embodiments, aryl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, benzofuranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from the group consisting of: O, N, and S. The heteroaryl group, including each heteroatom, can be unsubstituted or substituted with from 1 to 4 suitable substituents, as chemically feasible. For example, the heteroatom S may be substituted with one or two oxo groups, which may be shown as ═O. In some embodiments, heteroaryl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Alkoxy,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like. In some embodiments, alkoxy groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

An “amine” or “amino” is intended to mean the group —NH₂. “Optionally substituted” amines refers to —NH₂ groups wherein none, one or two of the hydrogens is replaced by a suitable substituent as described herein, such as alkyl, alkenyl, alkynyl, cycloalkyl, heterocycle, aryl, heteroaryl, alkoxy, carbonyl, carboxy, etc. In some embodiments, one or two of the hydrogens are optionally substituted with independently selected, halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy. Disubstituted amines may have substituents that are bridging, i.e., form a heterocyclic ring structure that includes the amine nitrogen.

An “amide” as used herein refers to an organic functional group having a carbonyl group (C═O) linked to a nitrogen atom (N), or a compound that contains this group, generally depicted as:

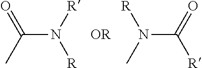

wherein, R and R′ can independently be any covalently-linked atom or atoms, for example, H, halo, alkyl, alkenyl, alkynyl, cycloalkvl, heterocycle, aryl, or heteroaryl.

A “urea” as used herein refers to a functional group having a carbonyl group (C═O) linked to a nitrogen atom (N), or a compound that contains this group, generally depicted as:

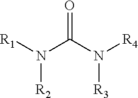

wherein, R₁, R₂, R₃, and R₄ can independently be any covalently-linked atom or atoms, for example, H, halo, alkyl, alkenyl, alkynyl, cycloalkyl, heterocycle, aryl, or heteroaryl.

A “thiourea” as used herein refers to a functional group the O atom of the carbonyl group in a urea is replaced by a S atom, generally depicted as:

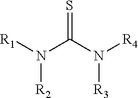

wherein, R₁, R₂, R₃, and R₄ can independently be any covalently-linked atom or atoms, for example, H, halo, alkyl, alkenyl, alkynyl, cycloalkyl, heterocycle, aryl, or heteroaryl.

A “thiol” or “mercapto” refers to an —SH group.

A “sulfide” or “thioether” as used herein refers to a group —S—R, where R is a suitable substituent, such as alkyl, cycloalkyl, heterocycle, aryl, or heteroaryl.

A “sulfone” as used herein refers to a sulfonyl functional group, generally depicted as:

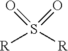

wherein, R can be any covalently-linked atom or atoms, for example, H, halo, alkyl, alkenyl, alkynyl, cycloalkyl, heterocycle, aryl, or heteroaryl.

A “sulfoxide” as used herein refers to a sulfinyl functional group, generally depicted as:

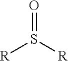

wherein, R can be any covalently-linked atom or atoms, for example, H, halo, alkyl, alkenyl, alkynyl, cycloalkyl, heterocycle, aryl, or heteroaryl.

A “tosyl”, “Tos”, and “Ts” as used herein refers to a tolulenesulfonyl functional group, generally depicted as:

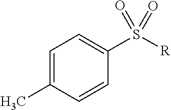

wherein, R can be any covalently-linked atom or atoms, for example, H, halo, alkyl, alkenyl, alkynyl, cycloalkyl, heterocycle, aryl, or heteroaryl.

A “triflate” and “OTf” as used herein refers to a trifluoromethanesulfonate functional group, generally depicted as:

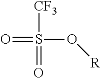

wherein, R can be any covalently-linked atom or atoms, for example, H, halo, alkyl, alkenyl, alkynyl, cycloalkyl, heterocycle, aryl, or heteroaryl.

“Carbonyl” is a functional group having a carbon atom double-bonded to an oxygen atom (C═O).

“Carboxy” as used herein refers to a —COOH functional group, also written as —CO₂H or —(C═O)—OH.

“Alkylation” is intended to mean a chemical reaction where an alkyl group is transferred from a reagent to a target molecule. Said alkyl group may be transferred as an alkyl carbocation, a free radical, a carbanion, or a carbene.

A “pharmaceutically acceptable salt” is intended to mean a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

A “pharmaceutically acceptable hydrate” or “pharmaceutically acceptable hydrated salt” is intended to mean a pharmaceutically acceptable salt of the definition herein that has one or more molecule of water included in its crystalline lattice.

“Form a ring” as used herein with respect to two substituents, e.g., R⁷ and R⁸ together forming a ring, refers to the two groups being linked together via one or more atoms (e.g., carbon) to form ring atoms making up a cycloalkyl, heterocyclo, aryl, or heteroaryl as described herein. Rings may be part of a monocyclic, bicyclic or tricyclic moiety, each of such ring being a saturated or unsaturated member of the monocyclic, bicyclic or tricyclic moiety.

“Radioisotope” or “radionuclide”, as used herein, refers to synthetic and/or naturally occurring atoms that have excess nuclear energy and where this excess energy is emitted as radiation. Examples of this type of radiation energy are alpha rays, beta rays, and gamma rays. Examples of radioisotopes include ^{11/13/14}C, ^{13/15}N, ¹⁵O, ¹⁸F, ^{44/47}Sc, ⁵⁵Co, ^{58m}Co, ⁶¹Cu, ⁶⁴Cu, ⁶⁷Cu, ⁶⁷Ga, ⁶⁸Ga, ⁷⁶Br, ⁷⁷Br, ⁸²Br, ⁸⁹Zr, ⁸⁷Y, ⁹⁰Y, ^{99m}Tc, ¹¹¹In, ¹²³I, ¹²⁴I, ¹²⁵I, ¹³¹I, ¹³⁴Ce, ¹³⁴La, ¹⁴⁹Tb, ¹⁵²Tb, ¹⁵⁵Tb, ¹⁵¹Tb, ¹⁷⁷Lu, ^{186/188}Re, ²⁰¹Tl, ²¹¹At, ²⁰³Pb, ²¹²Pb, ¹⁵³Sm, ⁸⁹Sr, ¹⁶⁶Ho, ¹⁷⁰Tm, ²¹²Bi, ²¹³Bi, ²²³Ra, ²²⁵Ac, ²²⁶Th, ²²⁷Th, and/or ²³⁰U.”

The terms “targeting ligand” or “targeting agent” are intended to mean a molecule with an affinity to bind, or be bound by, a specific tissue type and/or tumor type. In some embodiments, the affinity is due to the presence of a specific receptor protein or other molecule on the surface of cells in the tissue and/or tumor. In some embodiments, the targeting ligand will form a covalent or noncovalent attachment to the receptor protein or other molecule. Targeting ligands include, but are not limited to, a neurotensin receptor (NTSR1) ligand, a prostate-specific membrane antigen (PSMA) ligand, a fibroblast activation protein (FAP) inhibitor (FAPI), a C—X—C chemokine receptor type 4 (CXCR4) ligand, Bombesin (BBN), Arg-Gly-Asp (RGD), benzamide, folic acid and derivatives, lipids and derivatives, choline and derivatives, an amino acid, peptides, glucose and derivatives, melanin binding units, and other organic targeting molecules. In some embodiments, the targeting ligand is an antibody.

The terms “chelate”, “chelating agent”, or “chelator” are intended to mean a molecule with two or more functional groups that are able to donate at least two electron pairs and so bind a metal ion. It is common for the chelating agent to be an organic molecule. One chelating agent will often use its electron pairs to form a coordinate bond with one metal ion, though it is possible for a chelating agent to bond more than one metal ion. Examples of chelating compounds include, but are not limited to, dimercaptopropanol, ethylenediaminotetraacetic acid (EDTA), diethylenetriaminepentaacetic acid (DTPA), octadentate macrocyclic bifunctional 1,4,7,10-tetraazacyclododacane-1,4,7,10-tetraacetic acid (DOTA), DOTA-tert-butyl ester (M-120), 1,4,7,10-tetraazacyclododecane-1,4,7-triacetic acid trisodium salt (DO3A), hexadentate macrocyclic bifunctional 1,4,7-triazacyclononane-1,4,7-triacetic acid (NOTA), 2-S-(4-isothiocyanatobenzvl)-NOTA (NOTA-NCS), hydroxyethylidene diphosphonic acid (HEDP), ethylenediamine-N,N,N′,N′-tetrakis(methvlenephosphonic acid) (EDTMP), 1,4,7,10-tetraazacyclododecane-1,4,7,10-tetraaminomethylenephosphonic acid (DOTMP), mercaptoacetyltriglycine (MAG₃), Sar cage, cross-bridged macrocyclic chelators (e.g., CB-Cyclam or CB-TE2A), salicylic acid, triethanolamine, ferrioxamines, macropa and derivatives, and ionophores.

The term “therapeutic agent” is intended to mean a molecule that will, when provided to a subject in a therapeutically effective amount, provide some improvement or benefit to the subject. Those skilled in the art will appreciate that the therapeutic effects need not be complete or curative, as long as some benefit is provided to the subject. In some embodiments, the therapeutic agent may be a chemotherapeutic agent. Examples of a therapeutic agent include, but are not limited to, a poly(ADP-ribose) polymerase (PARP) inhibitor, an epidermal growth factor receptor (EGFR) inhibitor, a NTSR1 inhibitor (e.g., SR142948A), a tropomyosin receptor kinase (Trk) inhibitor, a human carbonic anhydrase IX (hCA) inhibitor, ado-trastuzumab emtansine (T-DM1), Paclitaxel (PTX), Doxorubicin (Dx), polypeptides, siRNAs, and oligonucleotides. In some embodiments, the therapeutic agent may be an immunotherapeutic agent. In some embodiments, the immunotherapeutic agent may be a stimulator of interferon genes protein (STING) agonist, a programmed cell death protein 1 (PD1) or PD-L1 ligand, a cytotoxic T-lymphocyte associated antigen 4 (CTLA4) ligand, a T-cell immunoglobulin domain and mucin domain-3 (Tim-3) ligand, a T-cell immunoglobulin and immunoreceptor tyrosine-based inhibitory motif domain protein (TIGIT) ligand, a lymphocyte activation gene-3 (LAG-3) ligand, a nuclear receptor subfamily 2 group F member 6 (NR2F6) ligand, a V-set immunoregulatory receptor (VISTA) ligand, or a B and T lymphocyte attenuator (BTLA) ligand.

The term “open reactive group” as used herein is intended to mean an atom, group of atoms, or specific chemical structure covalently linked to a compound that is/are able to undergo a chemical reaction and form a covalent bond with a target molecule and/or peptide. Examples of open reactive groups include, but are not limited to, alkenyl, sulfoxide, sulfone, and amine. In some embodiments, the open reactive group is a bioorthogonal reactant. Examples of bioorthogonal reactants include, but are not limited to, cyclooctene (e.g., trans-cyclooctene (TCO)), tetrazine, azide, nitrone, oxime, hydrazone, and the like.

The term “cyclic peptide” is intended to mean a polypeptide sequence that is covalently linked together to form a ring structure. The amino acids in the sequence of the cyclic peptide can be the same or different amino acids. In some embodiments, the ring structure can be formed by chemical bonding between consecutive or non-consecutive amino acids within the polypeptide sequence. In some embodiments, the ring of the cyclic peptide can be formed between the first amino acid (i.e., C-terminal amino acid) of the polypeptide and the last amino acid (i.e., N-terminal amino acid) of the polypeptide. In some embodiments, the ring structure is formed by amide bond, a disulfide bond, a thioether, bond, or any other known and chemically stable bond between two different amino acids in the polypeptide sequence. In some embodiments, a cyclic peptide may be radiolabeled for imaging or therapeutic purposes. In some embodiments, a cyclic peptide may be an imaging agent. In some embodiments, a cyclic peptide may be a therapeutic agent.

The term “bicyclic peptide” is intended to be a subtype of a cyclic peptide and refers to a polypeptide sequence that is covalently linked together to form a structure with two, interconnected rings. The amino acids in the sequence of the bicyclic peptide can be the same or different amino acids. In some embodiments, the bicyclic ring structure can be formed by chemical bonding between consecutive or non-consecutive amino acids within the polypeptide sequence. In some embodiments, the bicyclic ring structure can be stabilized using a chemical scaffold. Examples of such chemical scaffolds include, but are not limited to, 1,3,5-trismethylbenzene, 1,3,5-tris(bromomethyl)benzene, and 3,5-bis(mercaptomethyl)benzoic acid. In some embodiments, specific amino acids can be used to connect to the chemical scaffold. An example of an amino acid used to connect to the chemical scaffold includes, but is not limited to, cysteine. In some embodiments, the bicyclic ring structure is formed by an amide bond, a disulfide bond, a thioether, bond, or any other known and chemically stable bond between two or more different amino acids in the polypeptide sequence. In some embodiments, a bicyclic peptide may be radiolabeled for imaging or therapeutic purposes. In some embodiments, a bicyclic peptide may be an imaging agent. In some embodiments, a bicyclic peptide may be a therapeutic agent.

The term “linker” is intended to mean a chemical group or molecule that is capable of linking together two or more of the same or different chemical groups or moieties. In some embodiments, the linker is positioned between, or flanked by, two groups, molecules, or moieties and connected to each one via a covalent bond, thus connecting the two. Linkers include, but are not limited to polyethylene glycol (PEG, [e.g., PEG₄, PEG₆, or PEG₈]), a sequence of PEG molecules, an alkyl, an alkoxy, an alkylsulfide, a sulfone, an amine, an alkylamino, an acyl, a carbonyl, a carboxylic acid, a methoxycarbonyl, a heterocycle, a heteroaryl, a polypeptide, a vinyl sulfone, an amino acid, a triazole, a sequence of amino acids, an organic molecule, a saturated or unsaturated branched or unbranched alkyl chain, an antibody-drug conjugate (ADC) linker, a cyanuric acid, a triazine, a Sar cage, or other chemical moiety. In some embodiments, a linker is a compound having the structure:

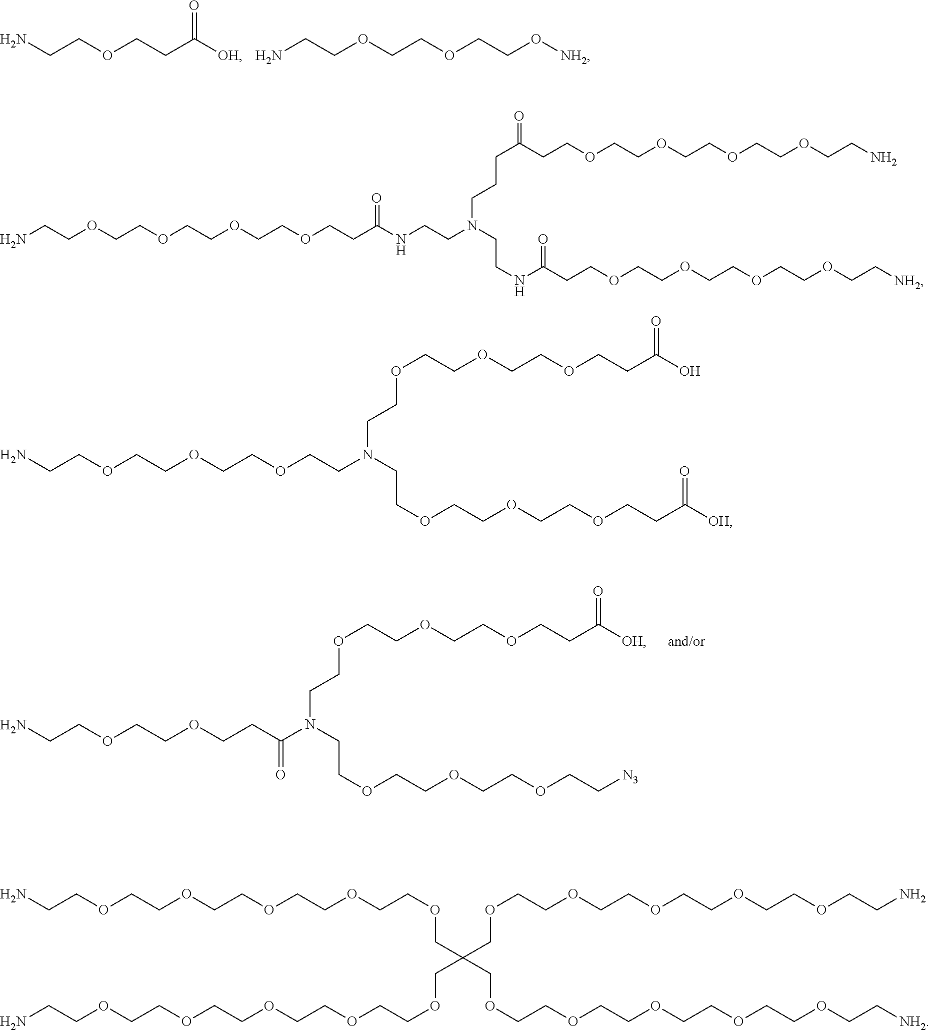

The terms “cancer” and “cancerous” are intended to mean the physiological condition typically characterized by unregulated cell growth in a portion of a multicellular organism. Often it is intended to mean a disease where the unregulated cell growth has the potential to spread and invade into multiple regions or tissues of the host organism (e.g., metastasis). A “tumor” comprises one or more cancerous cells. Examples of cancer include carcinoma, lymphoma, blastoma, sarcoma, melanoma, and leukemia or lymphoid malignancies.

The terms “radioactive label” or “radiotracer” are used herein to refer to a radioisotope that is used to generate an image that is detectable often using an appropriate instrument. Examples of techniques that use radiotracers include positron emission tomography (PET) and single photon emission computed tomography (SPECT). Examples of radiation emitted by radiotracers include gamma rays and X-rays.

The terms “radiation therapy” or “radiotherapy” are used herein to refer to a method of treating cancer whereby one or more tumors are destroyed using the radiation emitted from a radioisotope. The amount and delivery method of the radioisotope are often controlled in such a way as minimize harm to the host organism. Examples of radiation used in radiotherapy include alpha rays, beta rays, and Auger electrons.

The term “imaging agent” as used herein refers to any moiety useful for the detection, tracing, or visualization of a compound of the invention when coupled thereto. Imaging agents include, but are not limited to, an enzyme, a fluorescent dye (e.g., carbocyanine, indocarbocyanine, oxacarbocyanine, thiacarbocyanine, merocyanine, polymethine, coumarine, rhodamine, xanthene, fluorescein, boron-dipyrromethane, Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS), a fluorescent label, a luminescent label, a bioluminescent label, a magnetic label, a metallic particle (e.g., a gold particle), a nanoparticle, and a radioisotope. An imaging agent can be coupled to a compound of the invention by, for example, a covalent bond, ionic bond, van der Waals interaction or a hydrophobic bond. An imaging agent of the invention can be a radiolabel coupled to a compound of the invention, or a radioisotope incorporated into the chemical structure of a compound of the invention. Methods of detecting such imaging agents include, but are not limited to, positron emission tomography (PET), X-ray computed tomography (CT), magnetic resonance imaging (MRI), and single-photon emission computed tomography (SPECT).

The term “uptake” as used herein refers to the binding and/or absorption of a compound of the present invention by a cell, tissue, organ, and/or tumor. Uptake may occur through any mechanism familiar to one of skill in the art to which this invention belongs, such as endocytosis or active membrane transport or binding. The terms “low uptake” and “minimal uptake” are intended to mean uptake at concentrations too low to produce a therapeutic response and/or effect the normal function in the cell, tissue, organ, and/or tumor. In some embodiments, a compound with low uptake or minimal uptake in a cell, tissue, organ, and/or tumor will produce no detectable signal in said cell, tissue, organ, and/or tumor when viewed using any imaging method useful to the present invention. The term “high uptake” is intended to mean uptake at concentrations high enough to produce a therapeutic response and/or effect the normal function in the cell, tissue, organ, and/or tumor. In some embodiments, a compound of the present invention may have low uptake in one type of cell, tissue, organ, and/or tumor and high uptake in a different type of cell, tissue, organ, and/or tumor. In some embodiments, the amount of uptake of a compound of the present invention in two or more different types of cells, tissues, organs, and/or tumors may be compared in the form of a ratio of uptake between the two or more different types of cells, tissues, organs, and/or tumors.

### Active Compounds

Any targeting ligand that has preferential tumor accumulation would hold the potential to be used as radiotherapy agents once a proper nuclide is introduced. The compound cores presented herein demonstrate a versatile platform that can integrate PET, therapeutic radionuclides, fluorescent dyes, chemotherapeutics, and targeting ligands together in one compound. This platform is based on multifunctional (e.g., bifunctional, trifunctional, and/or tetrafunctional) heterocycle cores, including, without limitation, cyanuric acid, trithiocyanuric acid, triazine, barbituric acid, pyrimidine, Sar cages, DOTA, 1,10-diaza-18-crown-6, or alloxan. The compound structures presented here show that there are at least one, at least two, or at least three major functional groups that can be integrated together into one diagnostic and/or therapeutic compound. Example functional groups include, but are not limited to, chelating agents, radiolabels and/or radionuclides, targeting ligands (e.g., two of the same or two different), open reactive groups, cyclic peptides, fluorescent dyes or other imaging agents, and drugs (such as chemotherapeutic agents or immunotherapeutic agents). These different functional groups can be introduced onto the compound core by methods known in the art and as described herein. Example methods for attaching functional groups to the compound cores include alkylation, amide bond formation, reductive amination, nucleophilic substitution reaction, click chemistry, tetrazine-trans-cyclooctene (TTCO) ligation, urea bond formation, thiourea bond formation, phosphore bond formation, epoxide ring opening, and/or a reaction with thianthrenium salt (IT). Derivatives may also be prepared from cyanuric chloride, trichloroisocyanuric acid, melamine, or triazine.

Accordingly, one aspect of the invention provides a compound comprising a compound core which is a multifunctional (e.g., bifunctional, trifunctional, or tetrafunctional) heterocycle, wherein the compound core is covalently linked to at least one of a targeting ligand, a chelating agent, an imaging agent, a therapeutic agent, an open reactive group, a radionuclide, a cyclic peptide, or any combination thereof, or a pharmaceutically acceptable salt thereof.

In some embodiments, the compound core is covalently linked to one or more chelating agents, where each of the one or more chelating agents is covalently linked to a targeting ligand, an imaging agent, a therapeutic agent, an open reactive group, a radionuclide, and/or a cyclic peptide. In some embodiments, the compound core is covalently linked to two chelating agents (the same or different chelating agents), where each of the two chelating agents are independently linked to a targeting ligand, an imaging agent, a therapeutic agent, an open reactive group, a radionuclide, and/or a cyclic peptide. In some embodiments, the compound core is covalently linked to three chelating agents (the same or different chelating agents), where each of the three chelating agents are independently linked to a targeting ligand, an imaging agent, a therapeutic agent, an open reactive group, a radionuclide, and/or a cyclic peptide. In some embodiments, the compound core is covalently linked to four chelating agents (the same or different chelating agents), where each of the four chelating agents are independently linked to a targeting ligand, an imaging agent, a therapeutic agent, an open reactive group, a radionuclide, and/or a cyclic peptide. In some embodiments, the at least one homodimer or heterodimer covalently linked to the compound core comprises a chelating agent and a targeting ligand, an imaging agent, a therapeutic agent, an open reactive group, a radionuclide, or a cyclic peptide. In some embodiments, the compound has the structure:

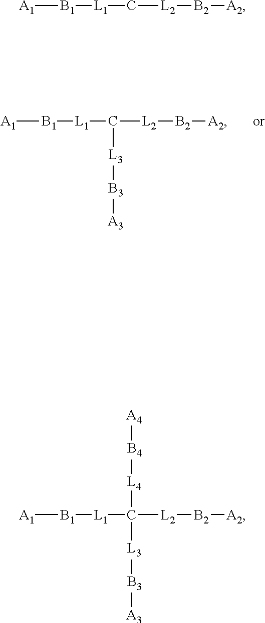

wherein C is the compound core;
L₁, L₂, L₃, and L₄ are each independently absent or a linker;
A₁, A₂, A₃, and A₄ are each independently a targeting ligand, an imaging agent, a therapeutic agent, an open reactive group, a radionuclide, or a cyclic peptide; and
B₁, B₂, B₃, and B₄, are each independently a chelator (the same or different chelators).

In some embodiments, the compound core is a multifunctional (e.g., bifunctional, trifunctional, or tetrafunctional) cyanuric acid, trithiocyanuric acid, triazine, barbituric acid, pyrimidine. Sar cage. DOTA, 1,10-diaza-18-crown-6, or alloxan, or a homodimer, a heterodimer, a homotrimer, a heterotrimer, a homotetramer, or a heterotetramer thereof.

In some embodiments, the compound core is covalently linked to at least two of a targeting ligand, a chelating agent, an imaging agent, a therapeutic agent, an open reactive group, a radionuclide, a cyclic peptide, or any combination thereof.

In some embodiments, the compound core is covalently linked to at least three of a targeting ligand, a chelating agent, an imaging agent, a therapeutic agent, an open reactive group, a radionuclide, a cyclic peptide, or any combination thereof.

In some embodiments, the compound core is covalently linked to at least four of a targeting ligand, a chelating agent, an imaging agent, a therapeutic agent, an open reactive group, a radionuclide, a cyclic peptide, or any combination thereof.

In some embodiments, the compound core is covalently linked to at a targeting ligand, a chelating agent, and an imaging agent. In some embodiments, the compound core is covalently linked to a targeting ligand, a chelating agent, and a radionuclide. In some embodiments, the compound core is covalently linked to a targeting ligand, a chelating agent, and a therapeutic agent. In some embodiments, the compound core is covalently linked to at a targeting ligand, an imaging agent, and a radionuclide. In some embodiments, the compound core is covalently linked to a targeting ligand, an imaging agent, and a therapeutic agent. In some embodiments, the compound core is covalently linked to a targeting ligand, a radionuclide, and a therapeutic agent. In some embodiments, the compound core is covalently linked to a targeting ligand, an open reactive group, and an imaging agent. In some embodiments, the compound core is covalently linked to a targeting ligand, an open reactive group, and a therapeutic agent. In some embodiments, the compound core is covalently linked to a targeting ligand, an open reactive group, and a chelating agent. In some embodiments, the compound core is covalently linked to a targeting ligand, an open reactive group, and radionuclide. In some embodiments, the compound core is covalently linked to at least two open reactive groups.

In some embodiments, at least two functional groups of the core are used to form a cyclic peptide. In some embodiments, the at least two open reactive groups bind one or more polypeptide to form a cyclic peptide. In some embodiments, the cyclic peptide is a bicyclic peptide.

In some embodiments, the compound core is covalently linked to at least two different targeting ligands. In some embodiments, the at least two different targeting ligands have affinity for different tissue types or two different targets on the same tissue type.

In some embodiments, the targeting ligand targets a tumor in a subject. In some embodiments, the targeting ligand is a neurotensin receptor (NTSR1) ligand, a prostate-specific membrane antigen (PSMA) ligand, a fibroblast activation protein (FAP) inhibitor (FAPI), a C—X—C chemokine receptor type 4 (CXCR4) ligand, Bombesin (BBN), Arg-Gly-Asp (RGD), benzamide, folic acid and derivatives, a lipid and derivatives, melanin binding ligands (e.g., chloroquine, hydroxychloroquine, phenothiazines, and the like), choline and derivatives, a peptide, or other organic targeting molecules. In some embodiments, the targeting ligand targets melanin (e.g., external melanin).

In some embodiments, the imaging agent is for optical imaging, PET imaging, or SPECT imaging. In some embodiments, the imaging agent is a fluorescent dye. In some embodiments, the fluorescent dye is selected from the group consisting of: carbocyanine, indocarbocyanine, cycloheptamethine cyanines, oxacarbocyanine, thiacarbocyanine, merocyanine, polymethine, coumarine, rhodamine, xanthene, fluorescein, boron-dipyrromethane, Cy5, Cy5.5, Cy7, indocyanine green (ICG), VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

In some embodiments, the chelating agent is selected from the group consisting of: dimercaptopropanol, ethylenediaminotetraacetic acid (EDTA), diethylenetriaminepentaacetic acid (DTPA), octadentate macrocyclic bifunctional 1,4,7,10-tetraazacyclododacane-1,4,7,10-tetraacetic acid (DOTA), hexadentate macrocyclic bifunctional 1,4,7-triazacyclononane-1,4,7-triacetic acid (NOTA), hydroxyethylidene diphosphonic acid (HEDP), ethylenediamine-N,N,N′,N′-tetrakis(methvlenephosphonic acid) (EDTMP), 1,4,7,10-tetraazacyclododecane-1,4,7,10-tetraaminomethylenephosphonic acid (DOTMP), mercaptoacetyltriglycine (MAG₃), salicylic acid, triethanolamine, ferrioxamine, macropa and derivatives, and ionophore.

In some embodiments, the chelating agent is bound to a radionuclide. In some embodiments, the radionuclide is for optical imaging, PET imaging, SPECT imaging, or radiotherapy. In some embodiments, the radionuclide is ¹¹C, ¹³N, ¹⁵O, ¹⁸F, ⁴⁴Sc, ⁵⁵Co, ^{58m}Co, ⁶¹Cu, ⁶⁴Cu, ⁶⁷Cu, ⁶⁷Ga, ⁶⁸Ga, ⁷⁶Br, ⁷⁷Br, ⁸²Br, ⁸⁹Zr, ⁸⁷Y, ⁹⁰Y, ^{99m}Tc, ¹¹¹In, ¹²³I, ¹²⁴I, ¹²⁵I, ¹³¹I, ¹³⁴Ce, ¹³⁴La, ¹⁴⁹Tb, ¹⁵²Tb, ¹⁵⁵Tb, ¹⁵¹Tb, ¹⁷⁷Lu, ¹⁸⁸Re, ²⁰¹Tl, ²¹¹At, ²⁰³Pb, ²¹²Pb, ²¹²Bi, ²¹³Bi, ²²³Ra, ²²⁵Ac, ²²⁶Th, ²²⁷Th, and/or ²³⁰U.

In some embodiments, the therapeutic agent is a chemotherapeutic agent. In some embodiments, the therapeutic agent is a poly(ADP-ribose) polymerase (PARP) inhibitor, an epidermal growth factor receptor (EGFR) inhibitor, a NTSR1 inhibitor (e.g., SR142948A), tropomyosin receptor kinase (Trk) inhibitor, human carbonic anhydrase IX (hCA) inhibitor, ado-trastuzumab emtansine (T-DM1), Paclitaxel (PTX), Doxorubicin (Dx), a polypeptide, a siRNA, and/or an oligonucleotide. In some embodiments, the therapeutic agent is afatinib, almonertinib, brigatinib, dacomitinib, erlotinib, gefitinib, icotinib, lapatinib, neratinib, olmutinib, osimertinib, pyrotinib, simotinib, and/or vandetanib.

In some embodiments, the therapeutic agent is an immunotherapeutic agent.

In some embodiments, the targeting ligand, chelating agent, imaging agent, therapeutic agent, open reactive group, radionuclide, and/or cyclic peptide are linked to the compound core by a linker. In some embodiments, the linker comprises a polyethylene glycol (PEG), an alkyl, an alkoxy, an alkylsulfide, a sulfone, an amine, an alkylamino, an acyl, a carbonyl, a carboxylic acid, a heterocycle, a heteroaryl, a polypeptide, and/or a vinyl sulfone.

One aspect of the invention provides a compound with the structure of Formula I:

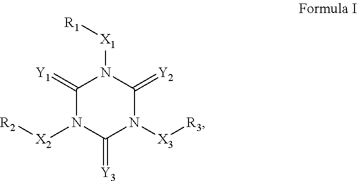

wherein R₁, R₂, and R₃ are independently absent, a targeting ligand, a chelating agent, an imaging agent, a therapeutic agent, an open reactive group, a radionuclide, a cyclic peptide, or any combination thereof; X₁, X₂, and X₃ are independently absent or a linker; and Y₁, Y₂, and Y₃ are independently O or S; or a pharmaceutically acceptable salt thereof.

In some embodiments, the compound of Formula I is a compound of Formula 1A:

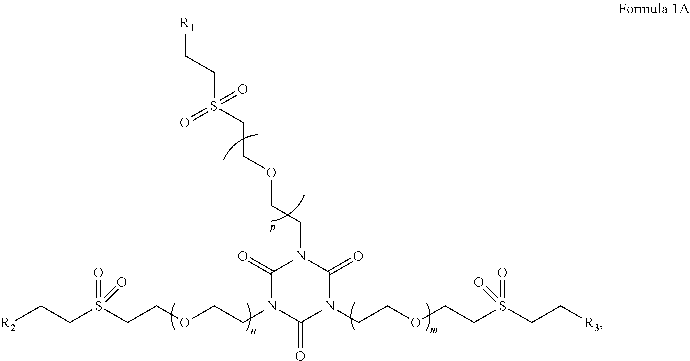

wherein R₁ is a chelating agent; R₂ and R₃ are independently a targeting ligand, an imaging agent, a therapeutic agent, an open reactive group, a radionuclide, a cyclic peptide, or any combination thereof; and n, m, and p are each independently an integer between 1 and 12; or a pharmaceutically acceptable salt thereof.

In some embodiments, the compound of Formula IA is Compound 1:

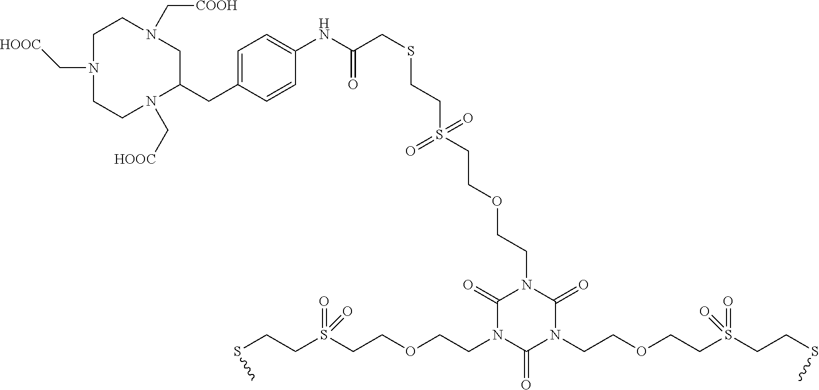

or a pharmaceutically acceptable salt thereof.

In some embodiments, the compound of Formula IA is Compound 2:

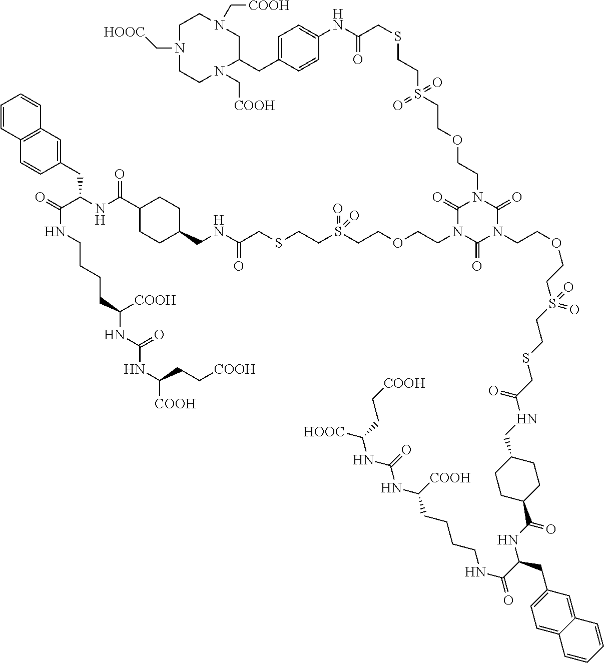

or a pharmaceutically acceptable salt thereof.

In some embodiments, the compound of Formula IA is:

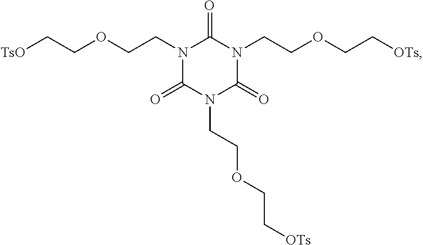

wherein Ts is a toluenesulfonyl (e.g., tosyl) group; or a pharmaceutically acceptable salt thereof.

In some embodiments, the compound of Formula IA is:

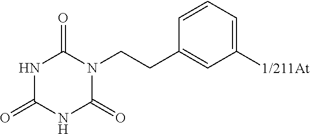

or a pharmaceutically acceptable salt thereof.

In some embodiments, the compound of Formula IA is:

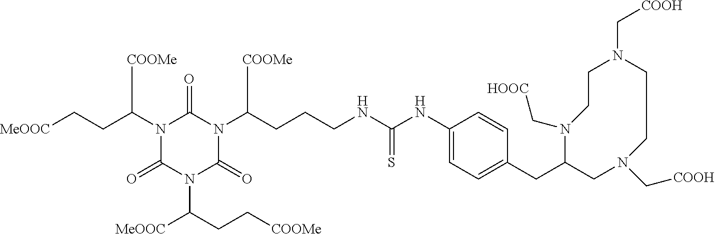 [SMILES: COC(=O)CCC(C(=O)OC)n1c(=O)n(C(CCCNC(=S)Nc2ccc(CC3CN(CC(=O)O)CCN(CC(=O)O)CCN3CC(=O)O)cc2)C(=O)OC)c(=O)n(C(CCC(=O)OC)C(=O)OC)c1=O]

or a pharmaceutically acceptable salt thereof.

In some embodiments, the compound of Formula IA is:

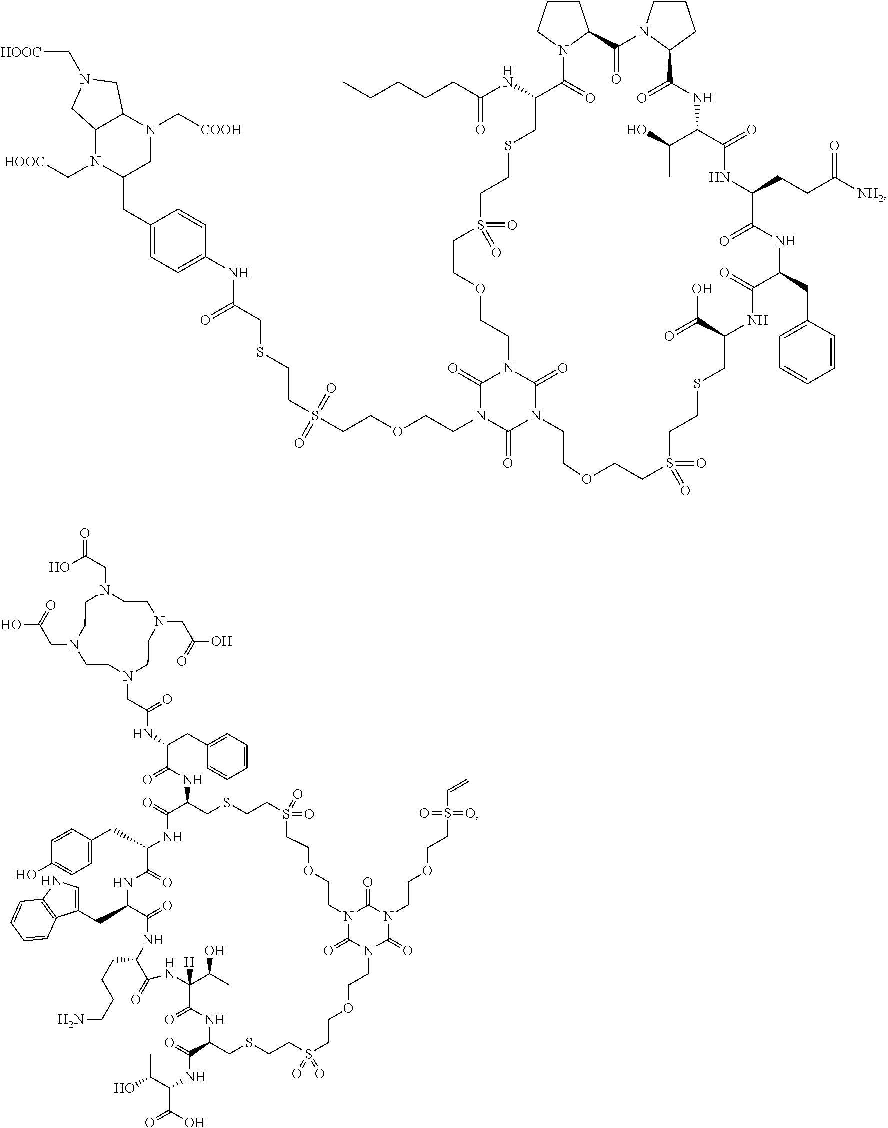

or a pharmaceutically acceptable salt thereof.

In some embodiments, the compound of Formula I is a compound of Formula IB:

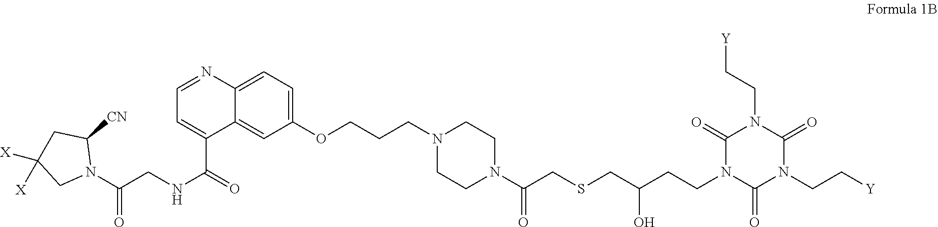

wherein X is H or F; and Y is an amine or a heterocycle; or a pharmaceutically acceptable salt thereof.

In some embodiments, the compound of Formula IB is Compound 3:

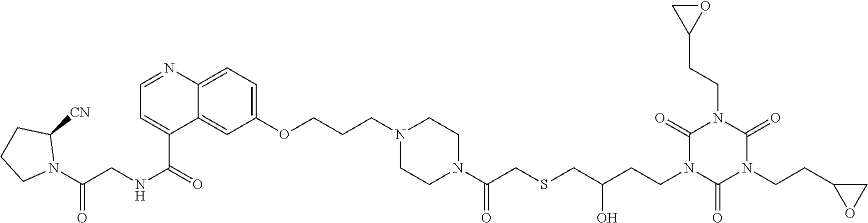 [SMILES: N#C[C@@H]1CCCN1C(=O)CNC(=O)c1ccnc2ccc(OCCCN3CCN(C(=O)CSCC(O)CCn4c(=O)n(CCC5CO5)c(=O)n(CCC5CO5)c4=O)CC3)cc12]

or a pharmaceutically acceptable salt thereof.

In some embodiments, the compound of Formula IB is Compound 4:

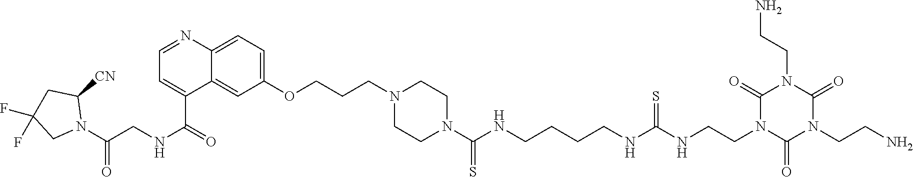 [SMILES: N#C[C@@H]1CC(F)(F)CN1C(=O)CNC(=O)c1ccnc2ccc(OCCCN3CCN(C(=S)NCCCCNC(=S)NCCn4c(=O)n(CCN)c(=O)n(CCN)c4=O)CC3)cc12]

or a pharmaceutically acceptable salt thereof.

Another aspect of the invention provides a compound with the structure of Formula II:

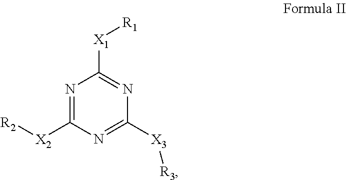

wherein R₁, R₂, and R₃ are independently absent, a targeting ligand, a chelating agent, an imaging agent, a therapeutic agent, an open reactive group, a radionuclide, a cyclic peptide, or any combination thereof; and X₁, X₂, and X₃ are independently absent or a linker; or a pharmaceutically acceptable salt thereof.

In some embodiments, the compound of Formula II is:

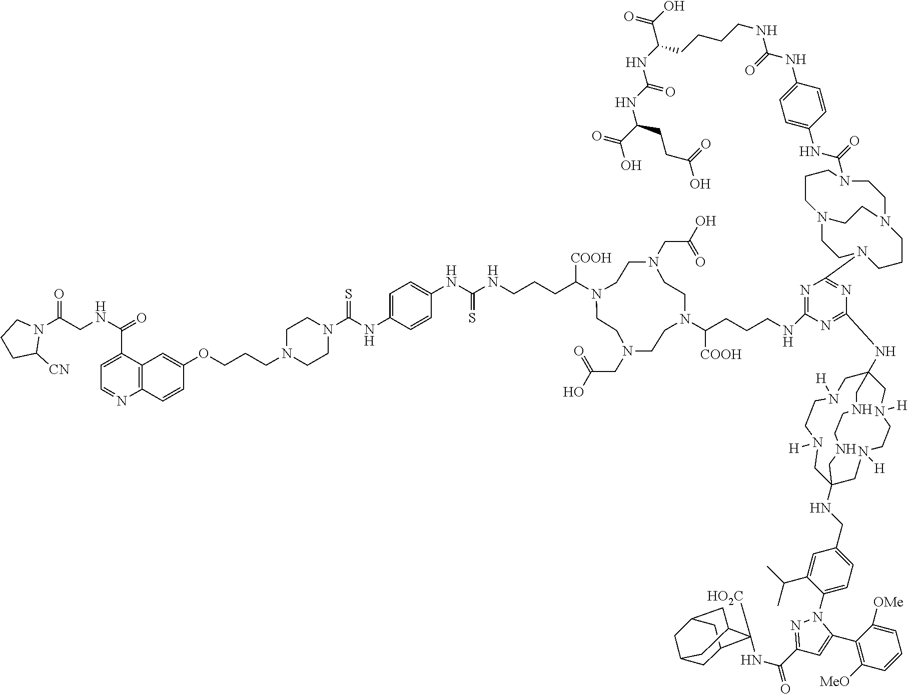 [SMILES: COc1cccc(OC)c1-c1cc(C(=O)NC2(C(=O)O)C3CC4CC(C3)CC2C4)nn1-c1ccc(CNC23CNCCNCC(Nc4nc(NCCCC(C(=O)O)N5CCN(CC(=O)O)CCN(C(CCCNC(=S)Nc6ccc(NC(=S)N7CCN(CCCOc8ccc9nccc(C(=O)NCC(=O)N%10CCCC%10C#N)c9c8)CC7)cc6)C(=O)O)CCN(CC(=O)O)CC5)nc(N5CCCN6CCN(CCCN(C(=O)Nc7ccc(NC(=O)NCCCC[C@H](NC(=O)N[C@@H](CCC(=O)O)C(=O)O)C(=O)O)cc7)CC6)CC5)n4)(CNCCNC2)CNCCNC3)cc1C(C)C]

or a pharmaceutically acceptable salt thereof.

In some embodiments, the compound of Formula II is a compound of Formula IIA:

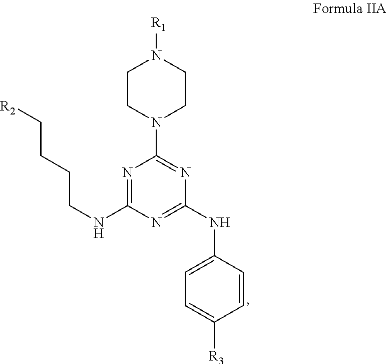

wherein R₁ is a therapeutic agent, R₂ is a targeting ligand, and R₃ is a chelating agent; or a pharmaceutically acceptable salt thereof.

In some embodiments, the compound of Formula IIA is Compound 5:

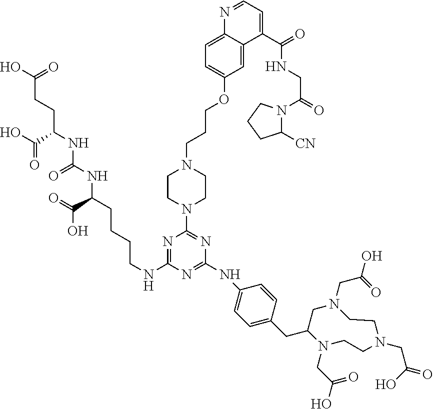 [SMILES: N#CC1CCCN1C(=O)CNC(=O)c1ccnc2ccc(OCCCN3CCN(c4nc(NCCCC[C@H](NC(=O)N[C@@H](CCC(=O)O)C(=O)O)C(=O)O)nc(Nc5ccc(CC6CN(CC(=O)O)CCN(CC(=O)O)CCN6CC(=O)O)cc5)n4)CC3)cc12]

or a pharmaceutically acceptable salt thereof.

In some embodiments, the compound of Formula IIA is Compound 6:

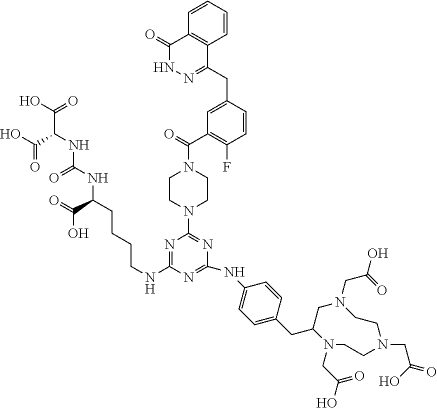

or a pharmaceutically acceptable salt thereof.

In some embodiments, the compound has the structure of Formula IIB

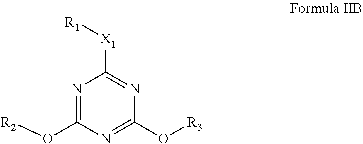

wherein R₁, R₂, and R₃ are independently absent, a targeting ligand, a chelating agent, an imaging agent, a therapeutic agent, an open reactive group, a radionuclide, a cyclic peptide, or any combination thereof; and X is absent or a linker; or a pharmaceutically acceptable salt thereof.

Another aspect of the invention provides a compound with the structure of Formula III:

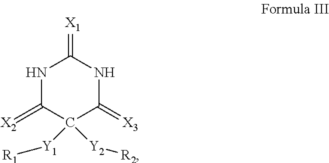

wherein X₁, X₂, and X₃ are independently O or S; R₁ and R₂ are independently absent, a targeting ligand, a chelating agent, an imaging agent, a therapeutic agent, an open reactive group, a radionuclide, a cyclic peptide, or any combination thereof; and Y₁ and Y₂ are independently absent or a linker; or a pharmaceutically acceptable salt thereof.

Another aspect of the invention provides a compound with the structure of Formula IV:

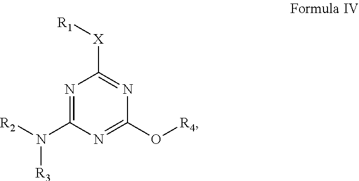

wherein X is O or S; and R₁, R₂, R₃, and R₄ are independently absent, a targeting ligand, a chelating agent, an imaging agent, a therapeutic agent, an open reactive group, a radionuclide, a cyclic peptide, or any combination thereof; or a pharmaceutically acceptable salt thereof.

Another aspect of the invention provides a compound with the structure of Formula V:

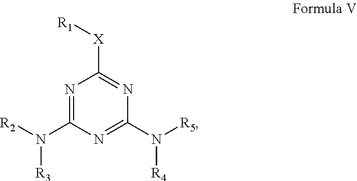

wherein X is O or S; and R₁, R₂, R₃, R₄, and R₅ are independently absent, a targeting ligand, a chelating agent, an imaging agent, a therapeutic agent, an open reactive group, a radionuclide, a cyclic peptide, or any combination thereof; or a pharmaceutically acceptable salt thereof.

Another aspect of the invention provides a compound with the structure of Formula VI:

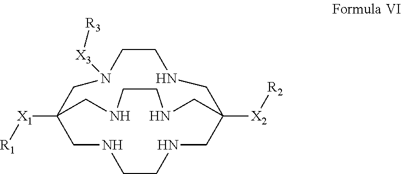

wherein R₁, R₂, and R₃ are independently absent, a targeting ligand, a chelating agent, an imaging agent, a therapeutic agent, an open reactive group, a radionuclide, a cyclic peptide, or any combination thereof; and X₁, X₂, and X₃ are independently absent or a linker; or a pharmaceutically acceptable salt thereof.

In some embodiments, the compound of Formula VI is Compound 7:

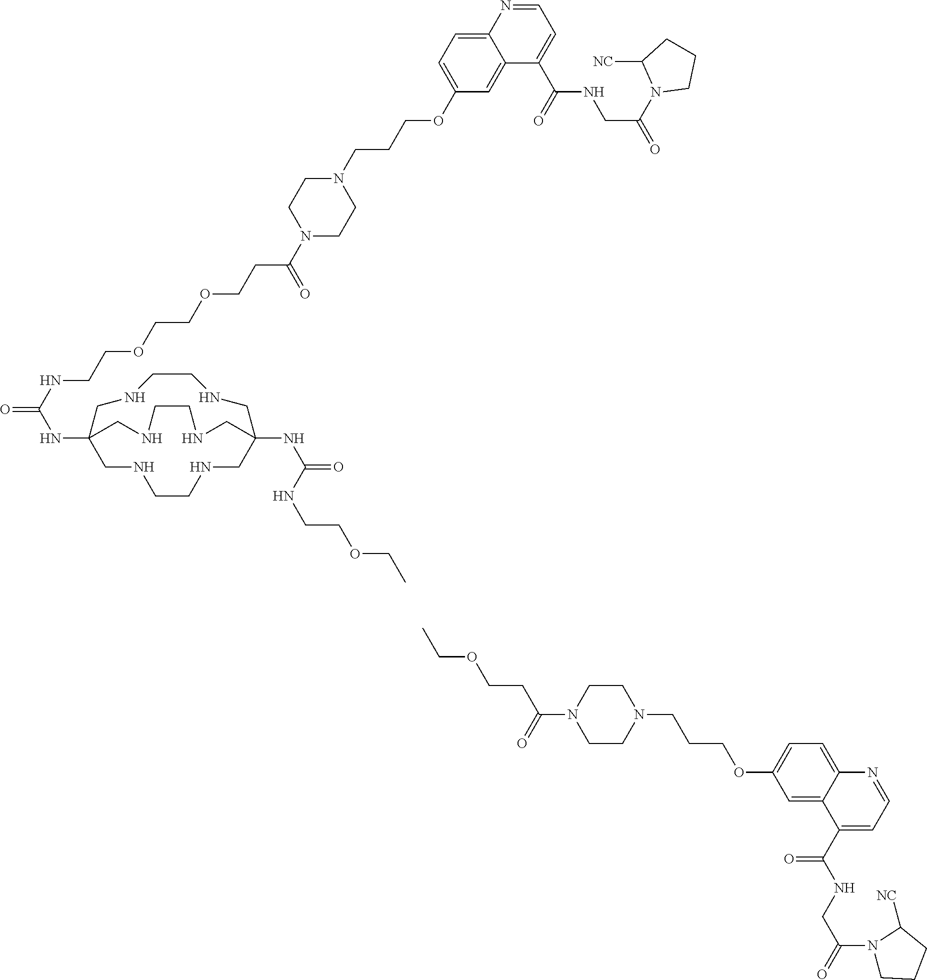 [SMILES: CCOCCNC(=O)NC12CNCCNCC(NC(=O)NCCOCCOCCC(=O)N3CCN(CCCOc4ccc5nccc(C(=O)NCC(=O)N6CCCC6C#N)c5c4)CC3)(CNCCNC1)CNCCNC2 ; CCOCCC(=O)N1CCN(CCCOc2ccc3nccc(C(=O)NCC(=O)N4CCCC4C#N)c3c2)CC1]

or a pharmaceutically acceptable salt thereof.

In some embodiments, the compound of Formula VI is

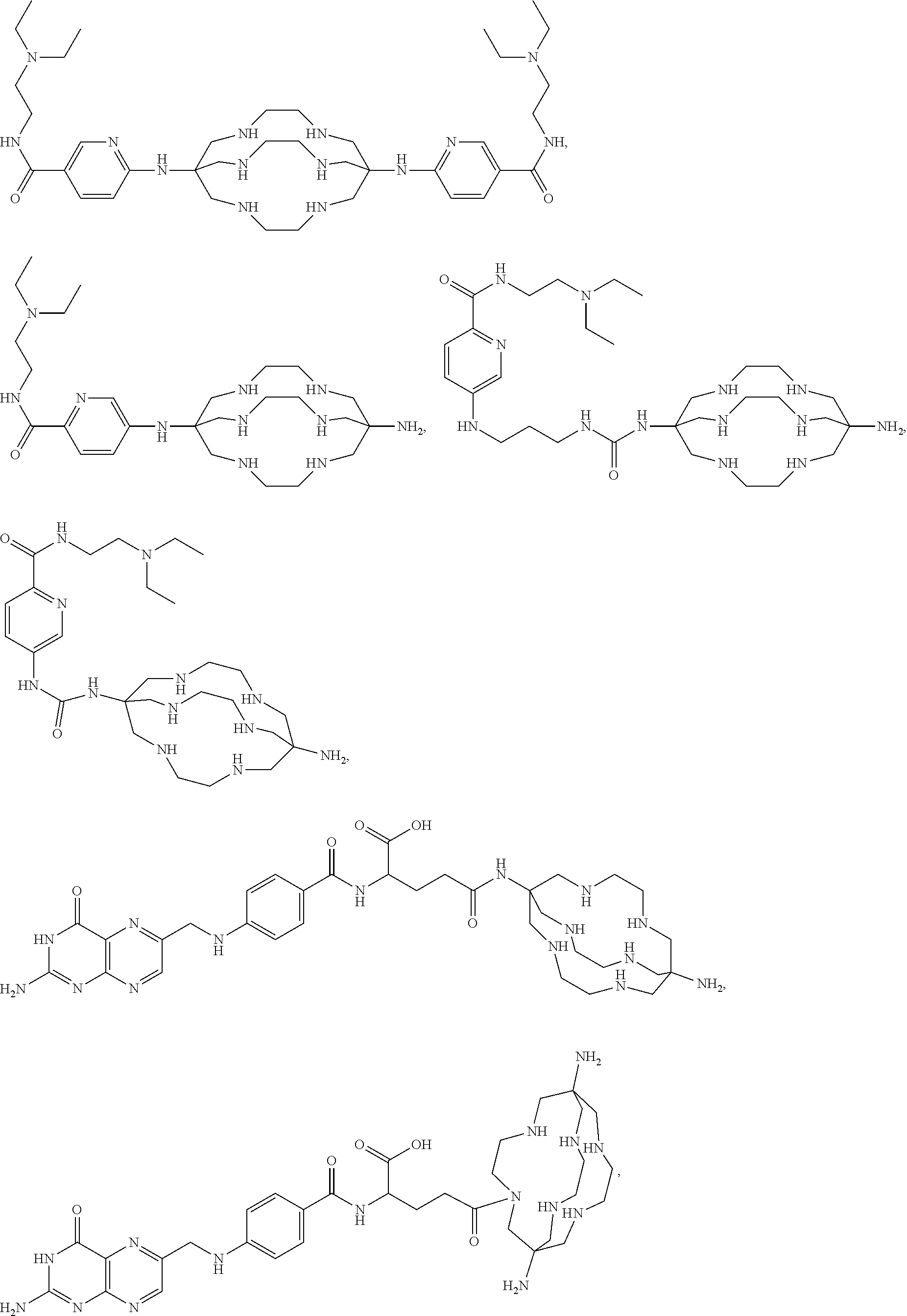 [SMILES: CCN(CC)CCNC(=O)c1ccc(NC23CNCCNCC(Nc4ccc(C(=O)NCCN(CC)CC)cn4)(CNCCNC2)CNCCNC3)nc1 ; CCN(CC)CCNC(=O)c1ccc(NC23CNCCNCC(N)(CNCCNC2)CNCCNC3)cn1 ; CCN(CC)CCNC(=O)c1ccc(NCCCNC(=O)NC23CNCCNCC(N)(CNCCNC2)CNCCNC3)cn1 ; CCN(CC)CCNC(=O)c1ccc(NC(=O)NC23CNCCNCC(N)(CNCCNC2)CNCCNC3)cn1 ; Nc1nc2ncc(CNc3ccc(C(=O)NC(CCC(=O)NC45CNCCNCC(N)(CNCCNC4)CNCCNC5)C(=O)O)cc3)nc2c(=O)[nH]1 ; Nc1nc2ncc(CNc3ccc(C(=O)NC(CCC(=O)N4CCNCC5(N)CNCCNCC(N)(CNCCNC5)C4)C(=O)O)cc3)nc2c(=O)[nH]1]

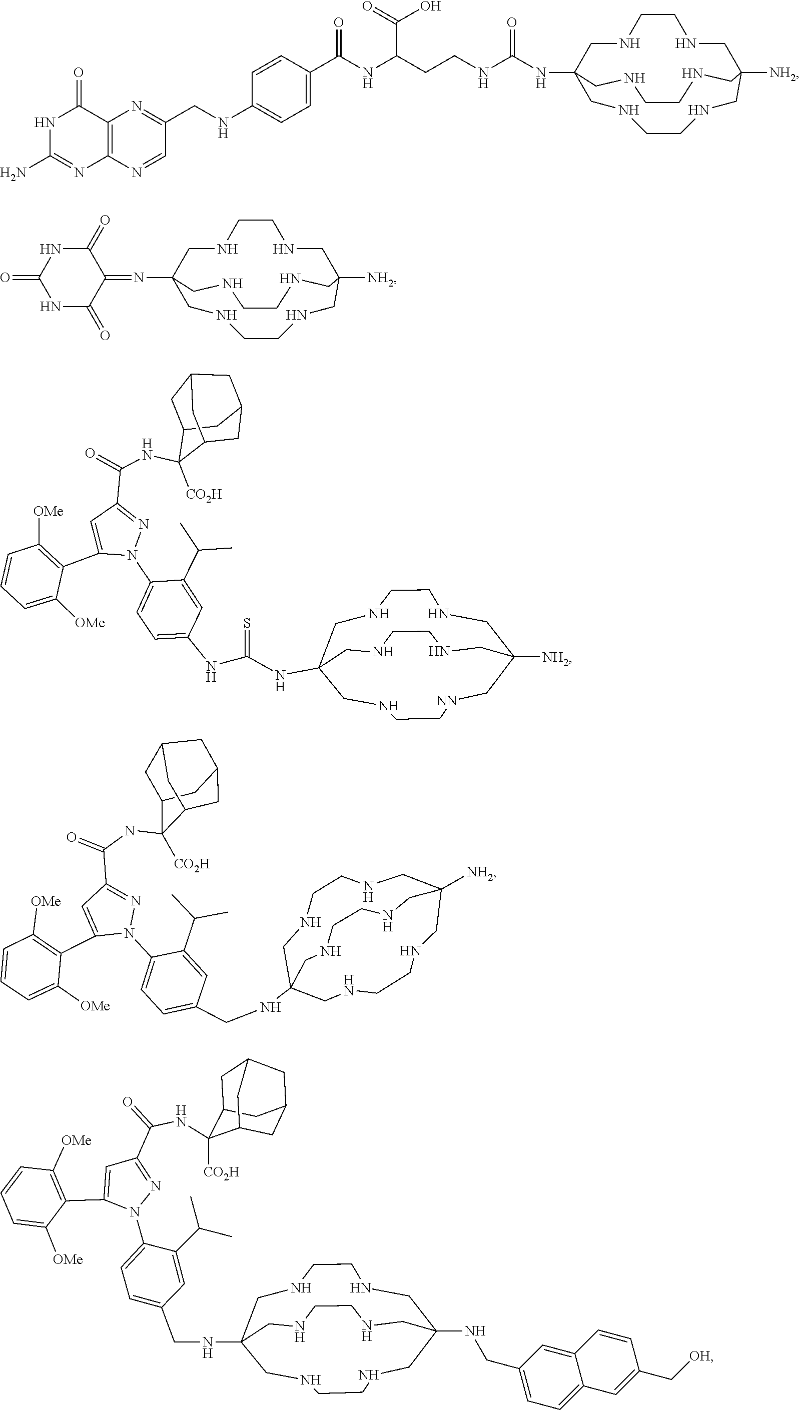

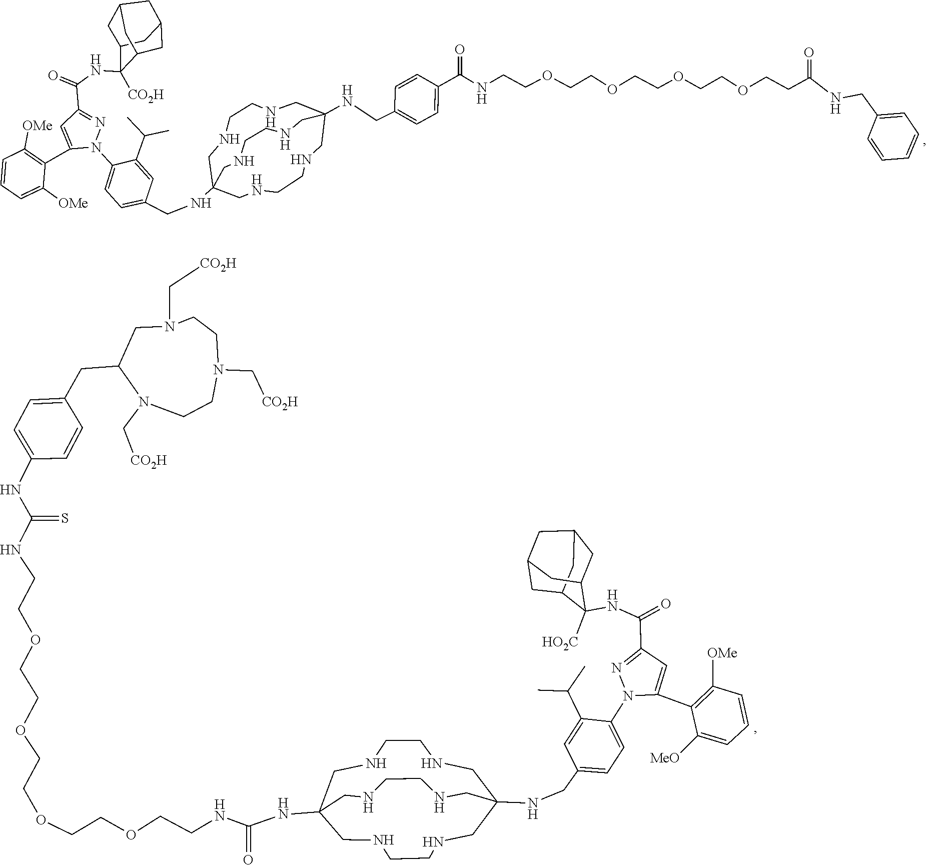 [SMILES: COc1cccc(OC)c1-c1cc(C(=O)NC2(C(=O)O)C3CC4CC(C3)CC2C4)nn1-c1ccc(CNC23CNCCNCC(NCc4ccc(C(=O)NCCOCCOCCOCCOCCC(=O)NCc5ccccc5)cc4)(CNCCNC2)CNCCNC3)cc1C(C)C ; COc1cccc(OC)c1-c1cc(C(=O)NC2(C(=O)O)C3CC4CC(C3)CC2C4)nn1-c1ccc(CNC23CNCCNCC(NC(=O)NCCOCCOCCOCCOCCNC(=S)Nc4ccc(CC5CN(CC(=O)O)CCN(CC(=O)O)CCN5CC(=O)O)cc4)(CNCCNC2)CNCCNC3)cc1C(C)C]

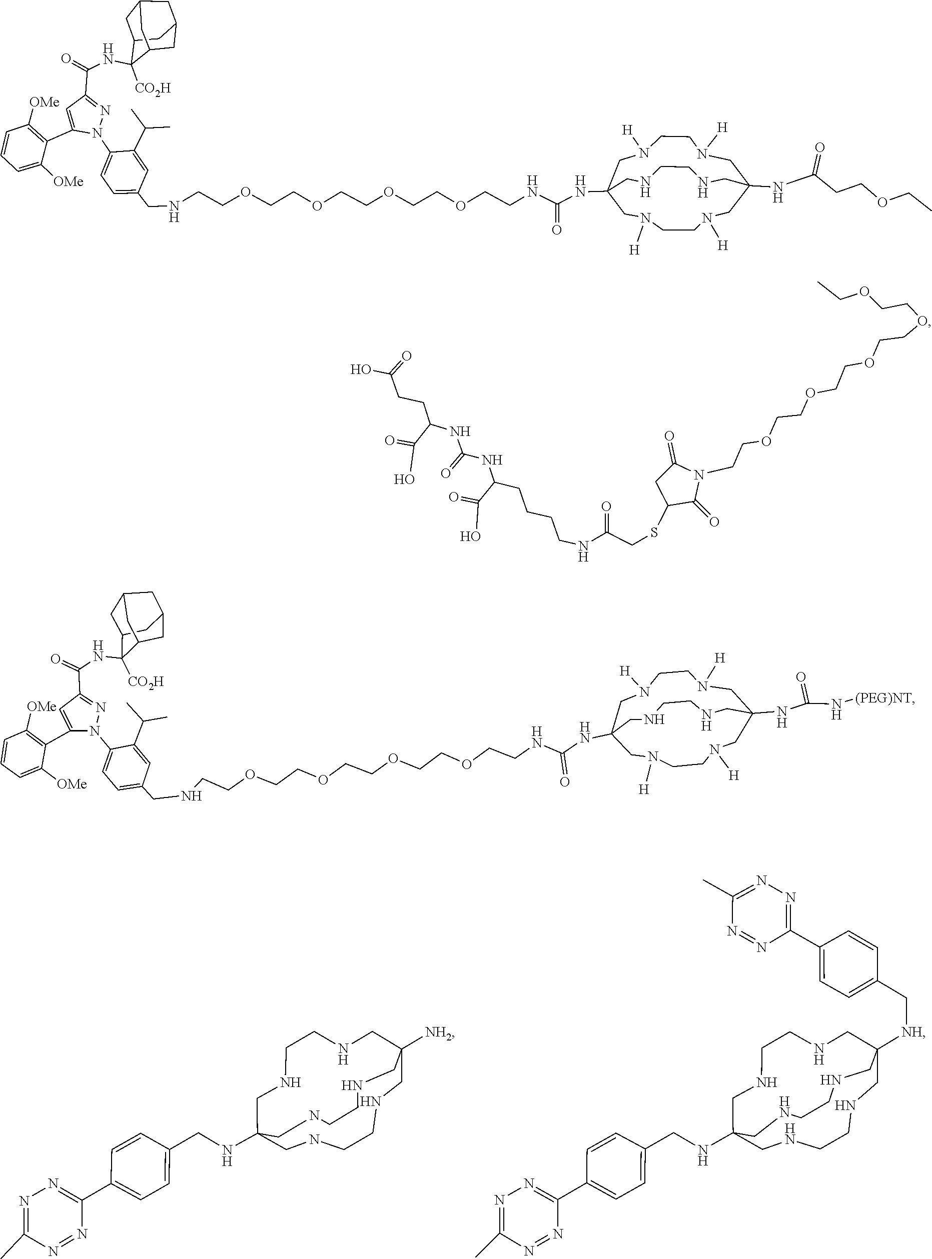

or a pharmaceutically acceptable salt thereof.

In some embodiments, the compound of Formula VI is

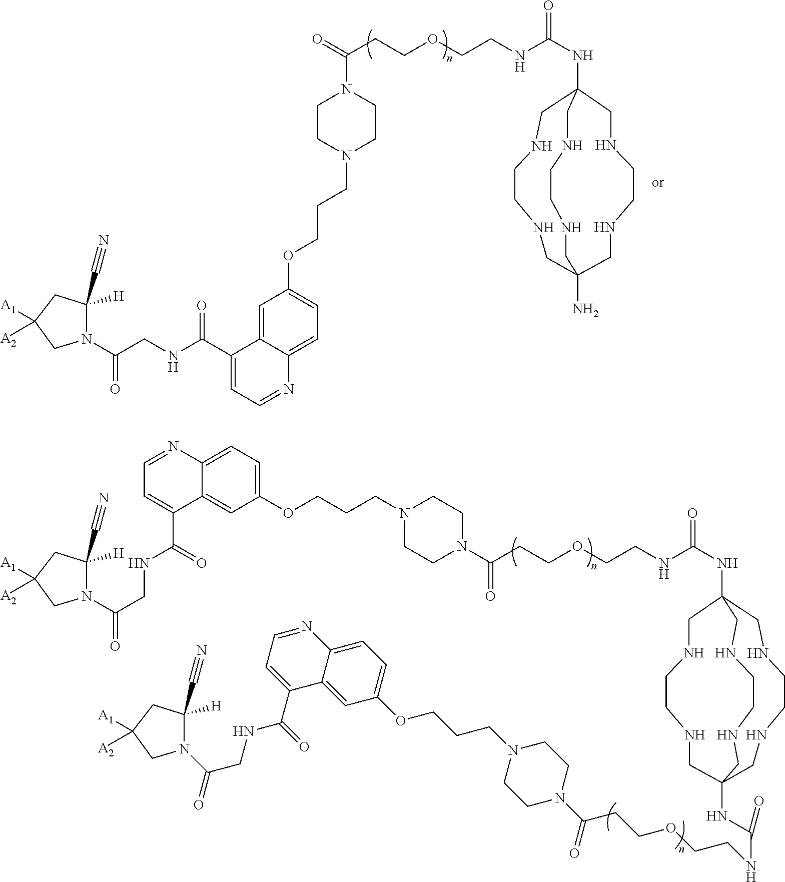

wherein A₁ and A₂ are each independently H or F, and each n is independently an integer between 1 and 12; or a pharmaceutically acceptable salt thereof.

In some embodiments, the compound of Formula VI is a compound with the structure of Formula VIA:

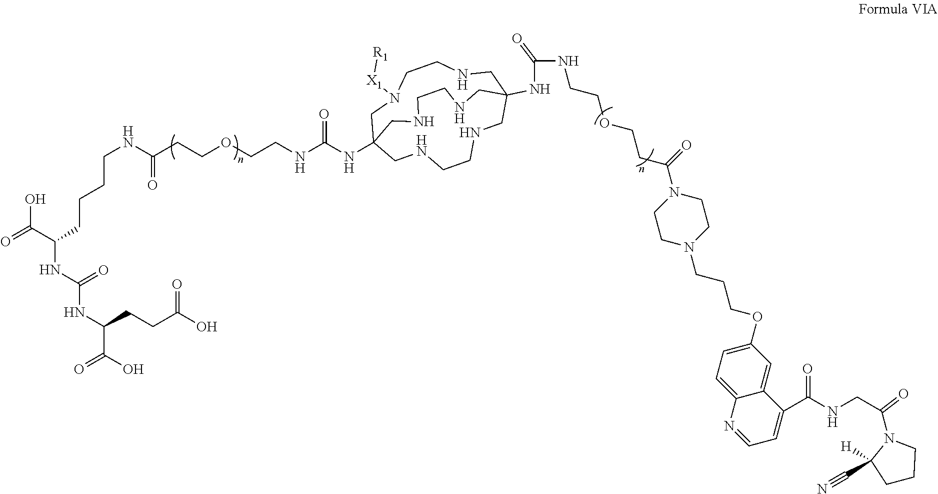

wherein R₁ is absent, a targeting ligand, a chelating agent, an imaging agent, a therapeutic agent, an open reactive group, a radionuclide, or a cyclic peptide; X₁ is absent or a linker; and each n is independently an integer from 1-12; or a pharmaceutically acceptable salt thereof.

In some embodiments, the compound of Formula VIA is

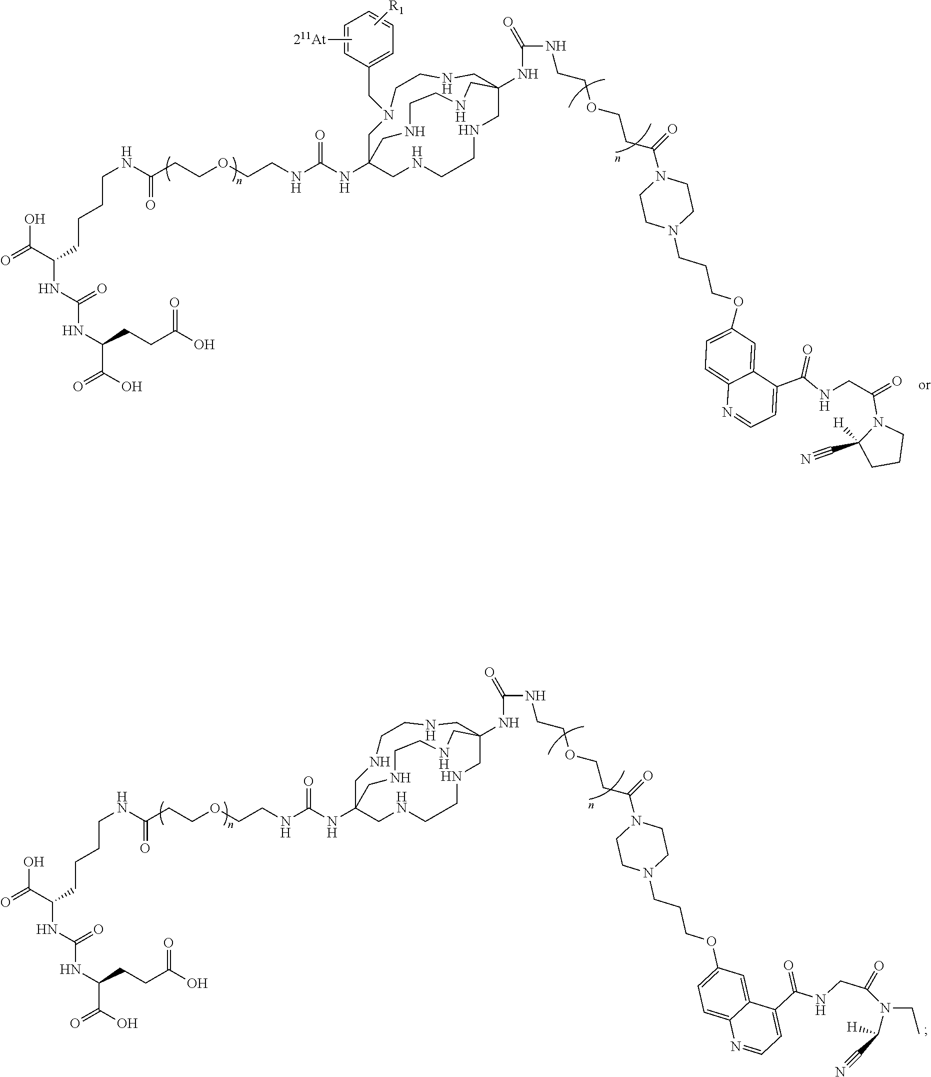

wherein each n is independently an integer from 1-12; or a pharmaceutically acceptable salt thereof.

In some embodiments, the compound of Formula VI is a compound with the structure of Formula VIB:

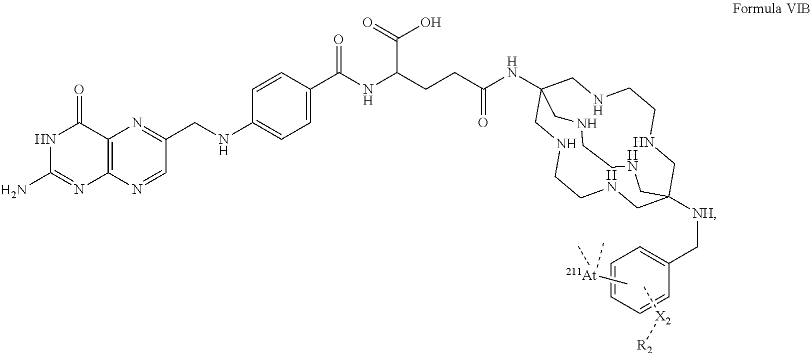

wherein R₂ is absent, a targeting ligand, a chelating agent, an imaging agent, a therapeutic agent, an open reactive group, a radionuclide, or a cyclic peptide; and X₂ is absent or a linker; or a pharmaceutically acceptable salt thereof.

Another aspect of the invention provides a compound with the structure of Formula VII:

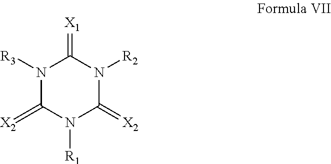

wherein R₁, R₂, and R₃ are independently absent, a targeting ligand, a chelating agent, an imaging agent, a therapeutic agent, an open reactive group, a radionuclide, a cyclic peptide, or any combination thereof; and X₁, X₂, and X₃ are independently O or S; or a pharmaceutically acceptable salt thereof.

Another aspect of the invention provides a compound with the structure of Formula VIII:

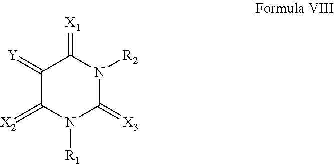

wherein R₁ and R₂ are independently absent, a targeting ligand, a chelating agent, an imaging agent, a therapeutic agent, an open reactive group, a radionuclide, a cyclic peptide, or any combination thereof; X₁, X₂, and X₃ are independently O or S; and Y is O or amine; or a pharmaceutically acceptable salt thereof.

Another aspect of the invention provides a compound with the structure of Formula IX:

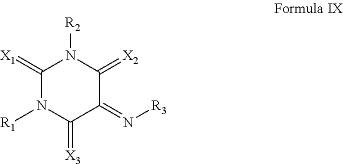

wherein X₁, X₂, and X₃ are independently O or S; R₁ and R₂ are independently absent, a targeting ligand, a chelating agent, an imaging agent, a therapeutic agent, an open reactive group, a radionuclide, a cyclic peptide, or any combination thereof; and R₃ is absent, a chelating agent, or a targeting ligand; or a pharmaceutically acceptable salt thereof.

In some embodiments, the compounds provided herein can be a heterodimer or a homodimer. In some embodiments, the heterodimer or homodimer is connected by one or more linkers as described herein. In some embodiments, the compounds provided herein can be a heterotrimer or a homotrimer. In some embodiments, the heterotrimer or homotrimer is connected by one or more linkers as described herein. In some embodiments, the compounds provided herein can be a heterotetramer or a homotetramer. In some embodiments, the heterotetramer or homotetramer is connected by one or more linkers as described herein. In some embodiments, each compound of the heterodimer, homodimer, heterotrimer, homotrimer, heterotetramer, and/or heterotetramer comprises a chelating agent (e.g., the same or different chelating agent) and further comprises a targeting ligand, an imaging agent, a therapeutic agent, an open reactive group, a radionuclide, and/or a cyclic peptide.

Another aspect of the invention provides a compound with the structure of Formula X:

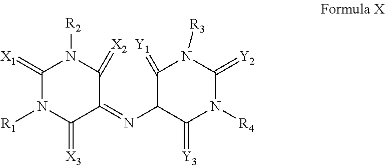

wherein X₁, X₂, X₃, Y₁, Y₂, and Y₃ are independently O or S; and R₁, R₂, R₃, and R₄ are independently absent, a targeting ligand, a chelating agent, an imaging agent, a therapeutic agent, an open reactive group, a radionuclide, a cyclic peptide, or any combination thereof; or a pharmaceutically acceptable salt thereof.

Another aspect of the invention provides a compound with the structure of Formula XI:

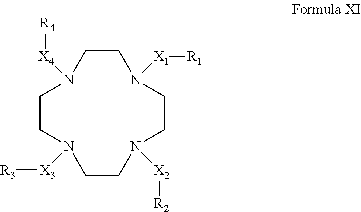

wherein R₁, R₂, R₃, and R₄ are independently absent, a targeting ligand, a chelating agent, an imaging agent, a therapeutic agent, an open reactive group, a radionuclide, a cyclic peptide, or any combination thereof; and X₁, X₂, X₃, X₄, are independently absent or a linker; or a pharmaceutically acceptable salt thereof.

In some embodiments, the compound of Formula XI is

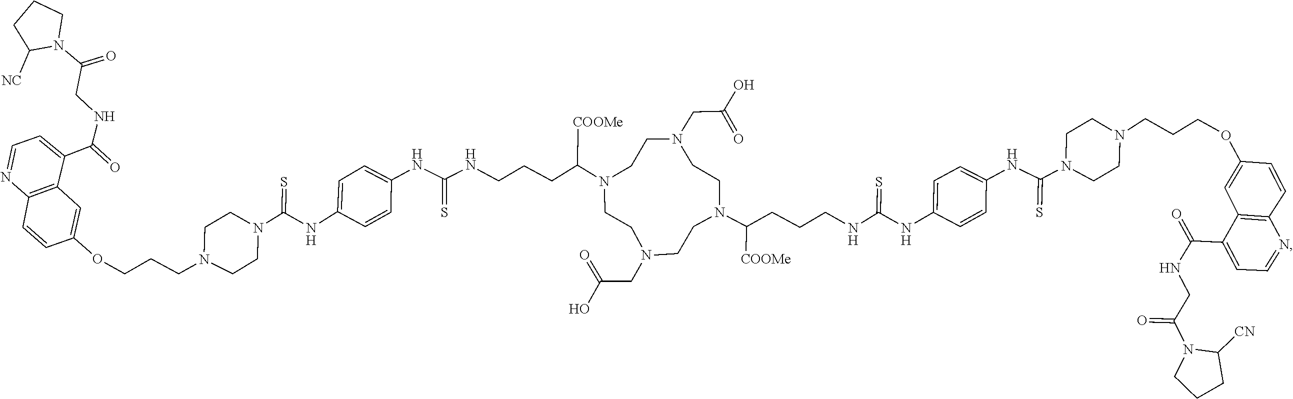 [SMILES: COC(=O)C(CCCNC(=S)Nc1ccc(NC(=S)N2CCN(CCCOc3ccc4nccc(C(=O)NCC(=O)N5CCCC5C#N)c4c3)CC2)cc1)N1CCN(CC(=O)O)CCN(C(CCCNC(=S)Nc2ccc(NC(=S)N3CCN(CCCOc4ccc5nccc(C(=O)NCC(=O)N6CCCC6C#N)c5c4)CC3)cc2)C(=O)OC)CCN(CC(=O)O)CC1]

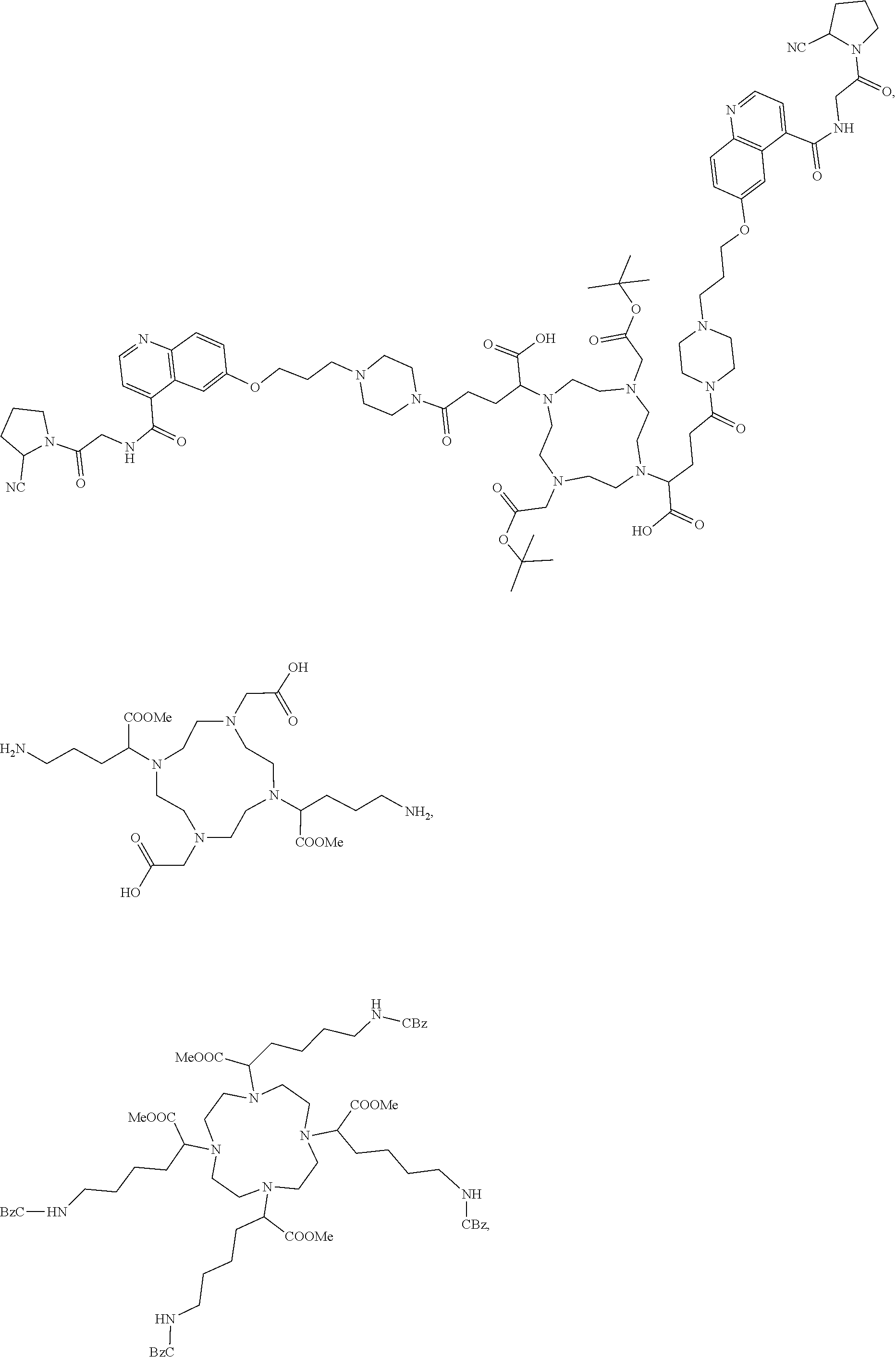

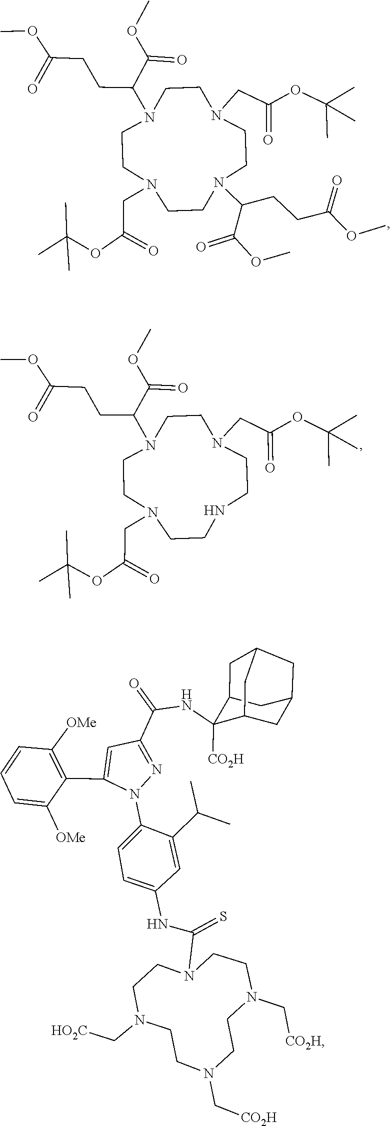

or a pharmaceutically acceptable salt thereof.

Another aspect of the invention provides a compound with the structure of Formula XII:

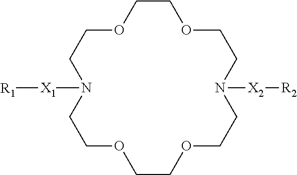

### Formula XII

wherein R₁ and R₂ are independently absent, a targeting ligand, a chelating agent, an imaging agent, a therapeutic agent, an open reactive group, a radionuclide, a cyclic peptide, or any combination thereof; and X₁ and X₂ are independently absent or a linker; or a pharmaceutically acceptable salt thereof.

In some embodiments, the compound of Formula XII is

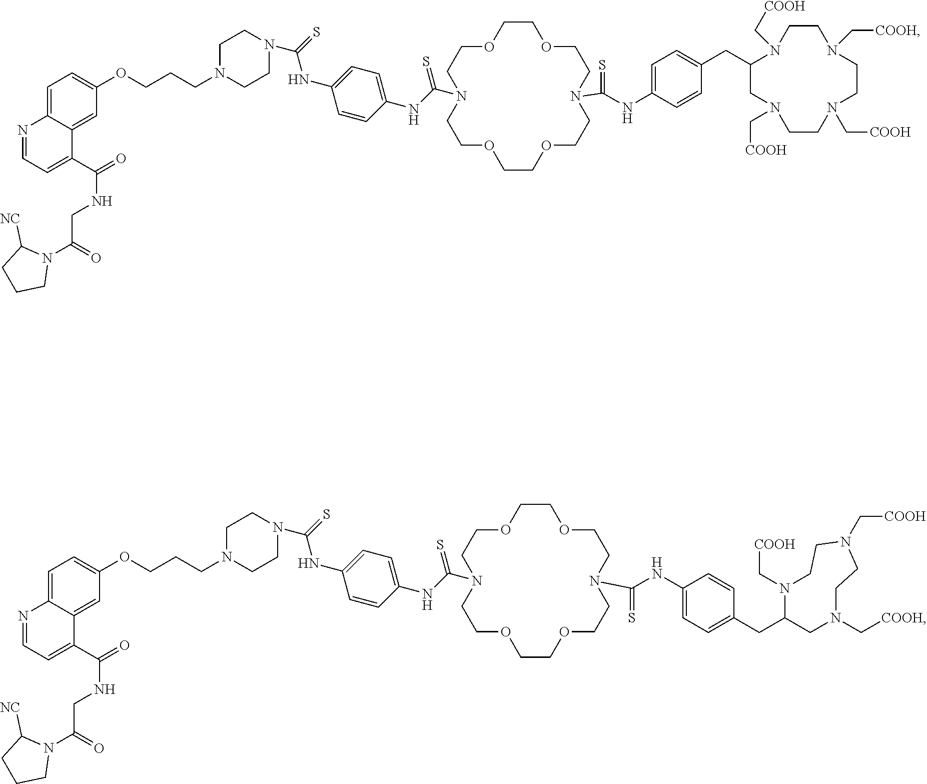 [SMILES: N#CC1CCCN1C(=O)CNC(=O)c1ccnc2ccc(OCCCN3CCN(C(=S)Nc4ccc(NC(=S)N5CCOCCOCCN(C(=S)Nc6ccc(CC7CN(CC(=O)O)CCN(CC(=O)O)CCN(CC(=O)O)CCN7CC(=O)O)cc6)CCOCCOCC5)cc4)CC3)cc12 ; N#CC1CCCN1C(=O)CNC(=O)c1ccnc2ccc(OCCCN3CCN(C(=S)Nc4ccc(NC(=S)N5CCOCCOCCN(C(=S)Nc6ccc(CC7CN(CC(=O)O)CCN(CC(=O)O)CCN7CC(=O)O)cc6)CCOCCOCC5)cc4)CC3)cc12]

or a pharmaceutically acceptable salt thereof.

Another aspect of the invention provides a compound with the structure of Formula XIII:

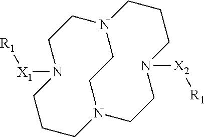

### Formula XIII

wherein R₁ and R₂ are independently absent, a targeting ligand, a chelating agent, an imaging agent, a therapeutic agent, an open reactive group, a radionuclide, a cyclic peptide, or any combination thereof; and X₁ and X₂ are independently absent or a linker; or a pharmaceutically acceptable salt thereof.

Another aspect of the invention provides a compound with the structure of Formula XIV:

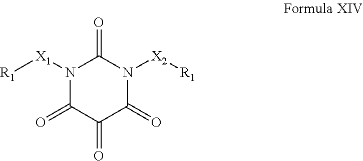

wherein R₁ and R₂ are independently absent, a targeting ligand, a chelating agent, an imaging agent, a therapeutic agent, an open reactive group, a radionuclide, a cyclic peptide, or any combination thereof; and X₁ and X₂ are independently absent or a linker; or a pharmaceutically acceptable salt thereof.

In some embodiments, the compound of Formula XIV is

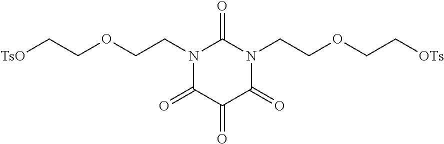 [SMILES: Cc1ccc(S(=O)(=O)OCCOCCN2C(=O)C(=O)C(=O)N(CCOCCOS(=O)(=O)c3ccc(C)cc3)C2=O)cc1]

or a pharmaceutically acceptable salt thereof.

In some embodiments, the compound is a Sar cage homodimer. In some embodiments, the Sar cage homodimer is a compound with the structure of XV:

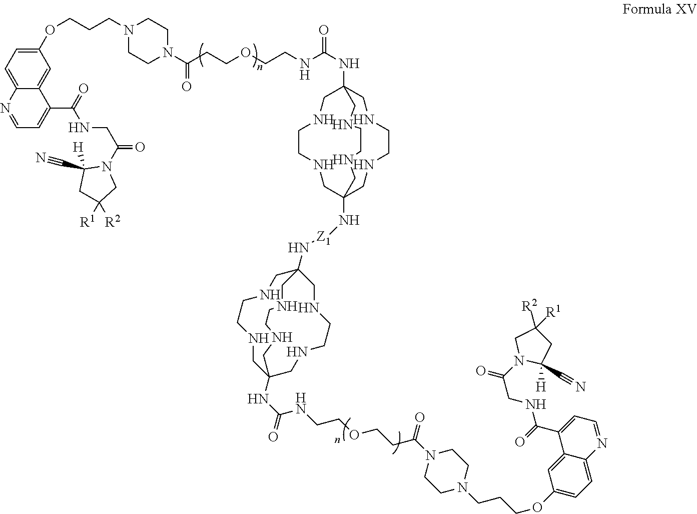

wherein R₁ and R₂ are independently H or F; Z₁ is a linker, and each n is independently an integer from 1 to 12; or a pharmaceutically acceptable salt thereof.

In some embodiments, the Sar cage homodimer is

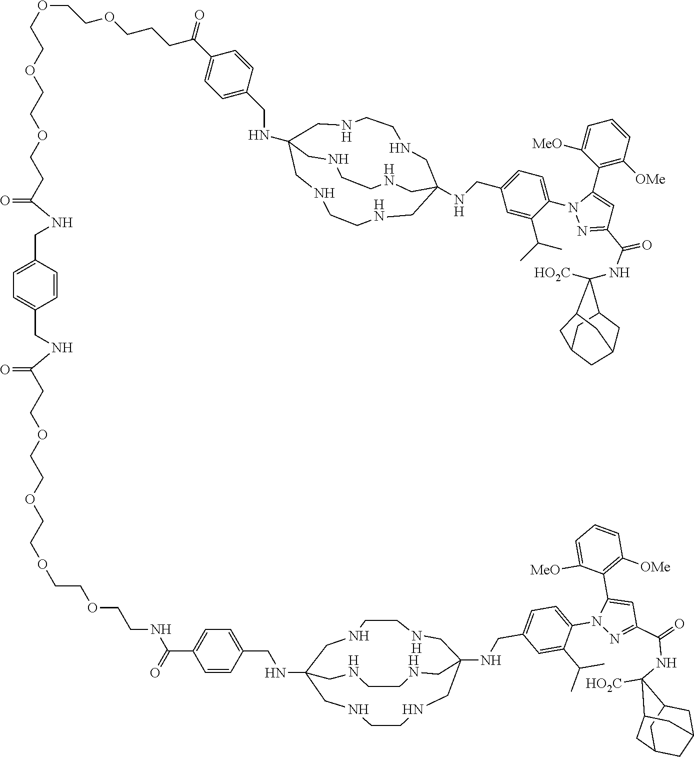 [SMILES: COc1cccc(OC)c1-c1cc(C(=O)NC2(C(=O)O)C3CC4CC(C3)CC2C4)nn1-c1ccc(CNC23CNCCNCC(NCc4ccc(C(=O)CCCOCCOCCOCCOCCC(=O)NCc5ccc(CNC(=O)CCOCCOCCOCCOCCNC(=O)c6ccc(CNC78CNCCNCC(NCc9ccc(-n%10nc(C(=O)NC%11(C(=O)O)C%12CC%13CC(C%12)CC%11C%13)cc%10-c%10c(OC)cccc%10OC)c(C(C)C)c9)(CNCCNC7)CNCCNC8)cc6)cc5)cc4)(CNCCNC2)CNCCNC3)cc1C(C)C]

or a pharmaceutically acceptable salt thereof

### Methods of Synthesis

Also provided herein is a method of synthesizing a compound, said method comprising attaching a targeting ligand, chelating agent, imaging agent, therapeutic agent, peptide reactive agent, and/or cyclic peptide to a compound core using, for example, alkylation, amide bond formation, reductive animation, nucleophilic substitution reaction, click chemistry, tetrazine-trans-cyclooctene (TTCO) ligation, urea bond formation, thiourea bond formation, phosphore bond formation, epoxide ring opening, and/or a reaction with thianthrenium salt (TT).

In some embodiments, the compound is prepared from a compound core, optionally the compound core is cyanuric acid, cyanuric chloride, trichloroisocyanuric acid, melamine, triazine, Sar cage, DOTA, 1,10-diaza-18-crown-6, alloxan, or any derivative thereof.

In some embodiments, the compound is prepared using the following reaction:

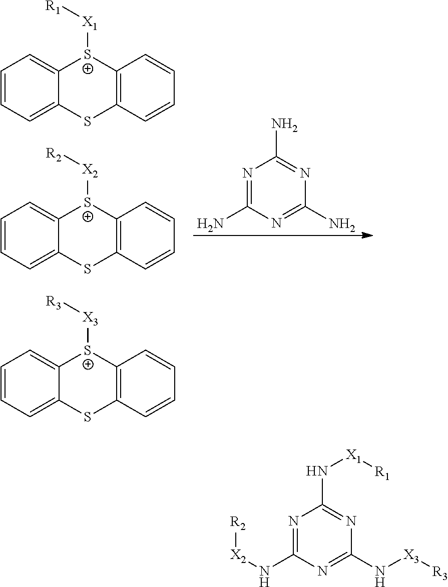

wherein R₁, R₂, and R₃ are independently a targeting ligand, a chelating agent, an imaging agent, a therapeutic agent, an open reactive group, a radionuclide, or a cyclic peptide; and X₁, X₂, X₃, and X₄ are independently absent or a linker. In some embodiments, the reaction ratio is controlled so that R₁, R₂, and R₃ are covalently attached to the compound core (e.g., melamine) in a 1:1:1 ratio. In some embodiments, the compound core (e.g., melamine) is reacted concurrently with the thiathrenium salts and the reaction product is purified to obtain a compound of the present invention. In some embodiments, the melamine is reacted sequentially with the thiathrenium salts and the reaction product is purified after each separate reaction.

In some embodiments, the compound is prepared using the following reaction:

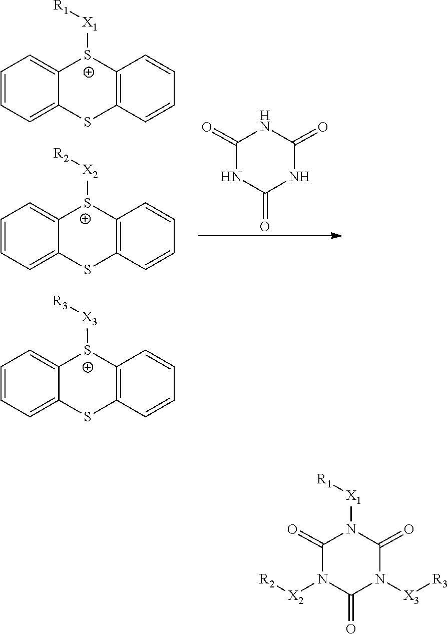

wherein R₁, R₂, and R₃ are each independently selected from a group consisting of a targeting ligand, a chelating agent, an imaging agent, a therapeutic agent, an open reactive group, a radionuclide, or a cyclic peptide; and X₁, X₂, and X₃ are each a linker. In some embodiments, the reaction ratio is controlled so that R₁, R₂, and R₃ are covalently attached to the compound core (e.g., cyanuric acid) in a 1:1:1 ratio. In some embodiments, the compound core (e.g., cyanuric acid) is reacted concurrently with the thiathrenium salts and the reaction product is purified to obtain a compound of the present invention. In some embodiments, the cyanuric acid is reacted sequentially with the thiathrenium salts and the reaction product is purified after each separate reaction.

In some embodiments, the reaction is carried out at room temperature (RT). In some embodiments, the reaction is heated from about 35 °C to about 200 °C (e.g., about 35 °C, 40 °C, 45 °C, 50 °C, 55 °C, 60 °C, 70 °C, 80 °C, 90 °C, 100 °C, 110 °C, 120 °C, 130 °C, 140 °C, 150 °C, 160 °C, 170 °C, 180 °C, 190 °C, or 200 °C). In some embodiments, the reaction is carried out in the presence of a solvent, e.g., dimethylformamide (DMF), water, dimethyl sulfoxide (DMSO), phosphate buffered saline (PBS), N,N-diisopropylethylamine (DIPEA), 1,8-diazabicyclo[5.4.0]undec-7-ene (DBU), triphenylphosphine (PPH₃), n-butanol acetic acid (NBAA), 1-butyl-1-methylpyrrolidinium trifluoromethanesulfonate (BmPy(OTf)), trifluoroacetic acid (TFA), triisopropyl silane (TIPS), phenol (PhOH), acetic acid, ethanol, potassium carbonate (K₂CO₃), sodium borohydride (NaBH₄), methanol (MeOH), ethanol, sodium triacetoxyborohydride (Na(OAC)3BH), ammonium acetate, acetic acid, and palladium on carbon (Pd—C). In some embodiments, the reaction can be carried out for about 1, 2, 3, 4, 5, 6, 7, 8, 9, 10, 11, 12, 13, 14, 15, 16, 17, 18, 19, 20, 21, 22, 23, or about 24 hours. In some embodiments, the reaction can be carried out overnight (o/n). In some embodiments, the reaction can be carried out for about 1, 2, 3, 4, 5, 6, or about 7 days.

### Methods of Use

Also provided herein is a method of detecting a tumor in a subject in need thereof, comprising administering to the subject a compound as described herein and detecting binding of the compound to the tumor, wherein said compound comprises at least one imaging agent, at least one chelating agent or radionuclide, and at least one targeting ligand that targets the tumor. In some embodiments, the tumor is a prostate cancer tumor. In some embodiments, the compound has low uptake into the salivary gland of the subject. In some embodiments, the compound has a high tumor to salivary gland uptake ratio. In some embodiments, the imaging agent is a cyclic peptide.

Also provided herein is a method of treating cancer in a subject in need thereof, comprising administering to the subject a therapeutically effect amount of a compound as described herein, thereby treating the cancer, wherein said compound comprises at least one therapeutic agent, at least one chelating agent or radionuclide, and at least one targeting ligand that targets the tumor. In some embodiments, the cancer is prostate cancer. In some embodiments, the compound has low uptake into the salivary gland of the subject. In some embodiments, the compound has a high tumor to salivary gland uptake ratio. In some embodiments, the therapeutic agent is a cyclic peptide. In some embodiments, the cancer is melanoma. In some embodiments, the compound has a high tumor to background ratio.

Also provided herein is a method of detecting and treating a tumor in a subject in need thereof, comprising: a first step of administering a compound as described herein to the subject and detecting the imaging agent, thereby determining the location and size of tumor, and a second step of administering a compound as described herein to said subject wherein said compound is provided in a therapeutically effective amount; and wherein said compound comprises at least one therapeutic agent, at least one imaging agent, at least one chelating agent or radionuclide, and at least one targeting ligand that targets the tumor. In some embodiments, the first step and the second step use the same compound. In some embodiments, the first step and the second step use different compounds. In some embodiments, the cancer is prostate cancer. In some embodiments, the compound has low uptake into the salivary gland of the subject. In some embodiments, the compound has a high tumor to salivary gland uptake ratio. In some embodiments, the imaging agent and/or therapeutic agent is a cyclic peptide.

Also provided herein is a method of detecting a tissue type in a subject, comprising administering to the subject a compound as described herein and detecting binding of the compound to the tissue type, wherein said compound comprises at least one imaging agent, at least one chelating agent or radionuclide, and at least one targeting ligand that targets the tissue type. In some embodiments, the imaging agent is a cyclic peptide.

In some embodiments, the compound does not cross the blood-ocular barrier (BOB). In some embodiments, the compound does not cross the blood-brain barrier (BBB). In some embodiments, the compound does not cross the blood-spinal cord barrier (BSCB).

Having described the present invention, the same will be explained in greater detail in the following examples, which are included herein for illustration purposes only, and which are not intended to be limiting to the invention.

### EXAMPLES

### Example 1

### Development of Novel PET Imaging and Radio/Chemo-Therapeutic Compounds for Prostate Cancer.

Prostate cancer is the most frequently diagnosed non-cutaneous malignancy and the second leading cause of cancer-related deaths among men in the US. Although various treatments have been developed, there is still an unmet need for greatly improved prostate cancer management due to its heterogeneity and the range of sensitivity to different treatment. Expressed in >85% of prostate cancers, Prostate Specific Membrane Antigen (PSMA) has emerged as a robust target for prostate cancer management recently. Moreover, PSMA was found to be further upregulated following treatment with antiandrogens in relapsed patients. Based on its specificity towards prostate cancer, various radiotracers have been developed for PSMA imaging and radiotherapy including ¹⁷⁷Lu and ²²⁵Ac. Despite the promising progress, various normal organs were also found to have high level of PSMA gene expression. This may not be a concern from imaging point of view, but it limits the therapeutic potential of targeted beta and alpha radionuclide therapies. Indeed, both preclinical and clinical data has suggested that PSMA targeted agents could have intense accumulation in kidneys and salivary glands (SGs). The kidney uptake of PSMA agents could be greatly reduced through ligand design or the administration of blocking agents. Thus, irradiation of SGs is the main dose-limiting side effect of radiolabeled PSMA-inhibitors, especially in PSMA-targeting beta and alpha radiotherapy.

Attempts to reduce SGs irradiation, such as sialendoscopy with dilatation, saline irrigation and steroid injection, only lead to limited benefit to the patient. In addition to non-specific uptake mechanisms. SGs physiologically express PSMA and so exhibit PSMA-specific uptake of small molecule PSMA-targeted agents. Consequently, it is challenging to design agents that only bind to tumor PSMA but not salivary gland PSMA, and any compounds that exhibit high tumor uptake without any concomitant SG uptake may improve patient outcomes for those suffering from prostate cancers.

Compounds with High Tumor Uptake and Greatly Reduced SG Uptake.

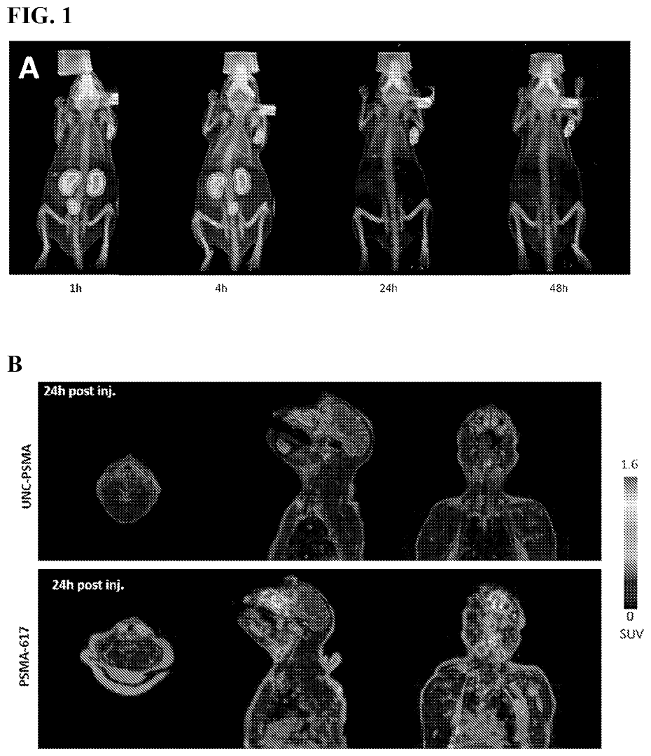

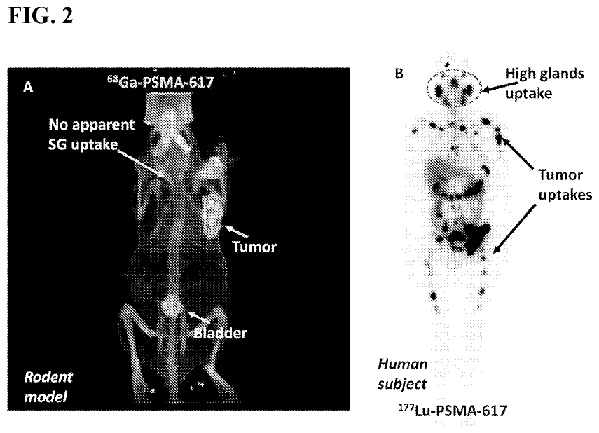

SPECT/CT imaging of treatments with the FDA approved ¹⁷⁷Lu-PSMA-617 demonstrated prominent SG uptake in both rodent models and in a human subject (FIG. 2, panels A and B). By comparison, the ⁶⁴Cu-NOTA-UNC-PSMA2 compound, shown below, demonstrated 2-3 times higher tumor uptake compared to PSMA-617 in both rodent models and non-human primate (NHP) models (FIG. 1, panels A and B). This distribution was persistent as only the tumor demonstrated prominent compound uptake at 24 and 48 h post injection (FIG. 1, panel A). Indeed, SGs uptake of ⁶⁴Cu-NOTA-UNC-PSMA2 is only 1/10th of ⁶⁴Cu-PSMA-617 at 24 h post-injection in the NHP study (FIG. 1, panel B).

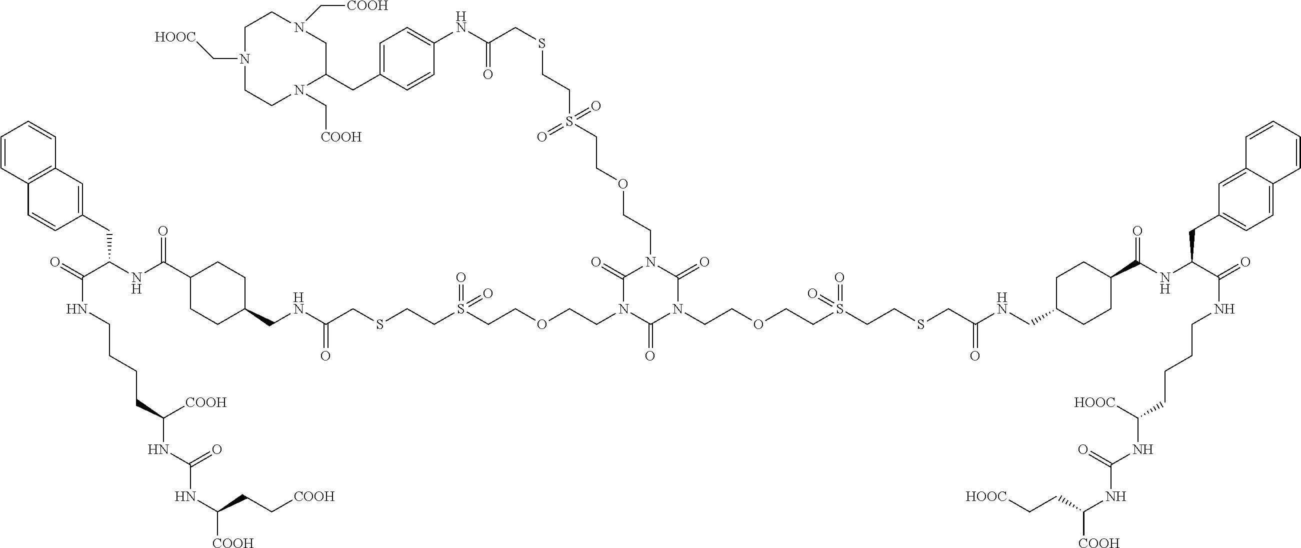

Example Compound Core Synthesis with Alkyl Thianthrenium Salts.

Cyanuric acid, melamine, and their derivatives are inexpensive and readily available compounds. Functionalization of these compounds, especially cyanuric acid, is challenging and requires reactive alkylating agents and/or high temperatures. The reactions below demonstrate the use of alkyl thianthrenium salts to functionalize cyanuric acid under mild conditions. These reactions are carried out typically in the presence of dimethylformamide and may be carried out at room temperature or while being heated. This functionalization reaction is also applicable to melamine derivatives and hybrid melamine/cyanuric acid derivatives. In some embodiments, more than one (e.g., two or more) functional groups are concurrently attached to the compound core in one functionalization reaction. In some embodiments, the functionalization reaction further comprises a purification step after the two or more of the functional groups are concurrently attached to the compound core. In some embodiments, more than one (e.g., two or more) functional groups are sequentially attached to the compound core in more than one (e.g., two or more) separate functionalization reactions. In some embodiments, each separate functionalization reaction further comprises a purification step between the addition of each functional group to the compound core. Any type of purification known to one skilled in the art may be used for the purification step, including, but not limited to, filtration, chromatography, distillation, and/or centrifugation.

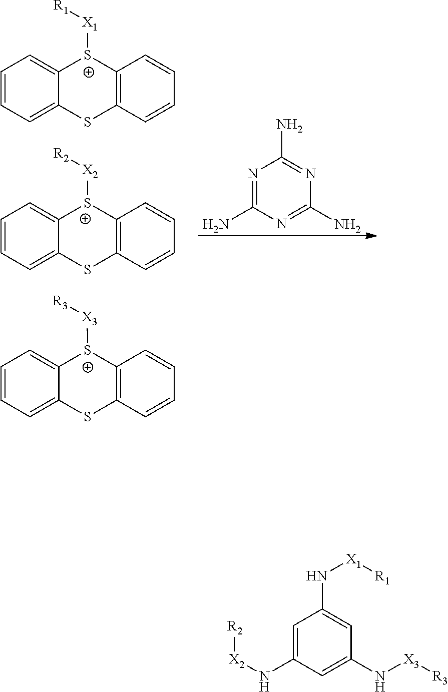

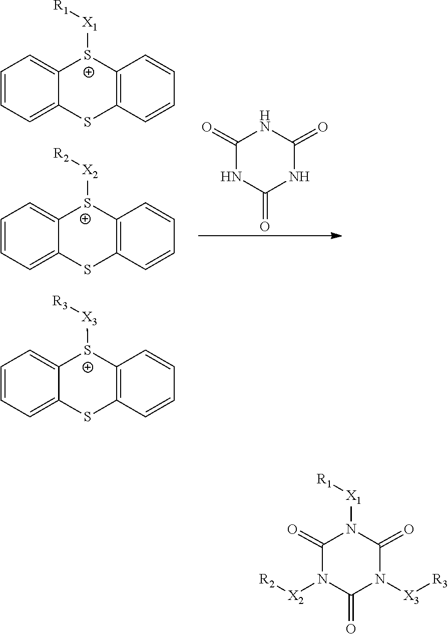

### Example Compound Synthesis.

Functional group attachment to the compound core can be carried out using the example reactions below. The compound core may be covalently linked with reactive groups to facilitate the attachment of functional groups. These reactive groups may be useful in alkylation, amide bond formation, reductive amination, nucleophilic substitution reactions, click chemistry, tetrazine-trans-cyclooctene (TTCO) ligation, urea bond formation, thiourea bond formation, phosphore bond formation, epoxide ring opening, and/or a reaction with thianthrenium salt (TT). Example reactive groups that facilitate this attachment include, but are not limited to, amine, acid, azide, alkyne, bicyclononyne (BCN), isocyanate (NCO), N-chlorosuccinimide (NCS), tetrazine, trans-cycloctene (TCO), maleimide, thiol, or epoxide. In other embodiments, the reactive groups may be used to anchor the compound at the binding site so tumor retention is longer.

Example synthesis schemes are shown below.

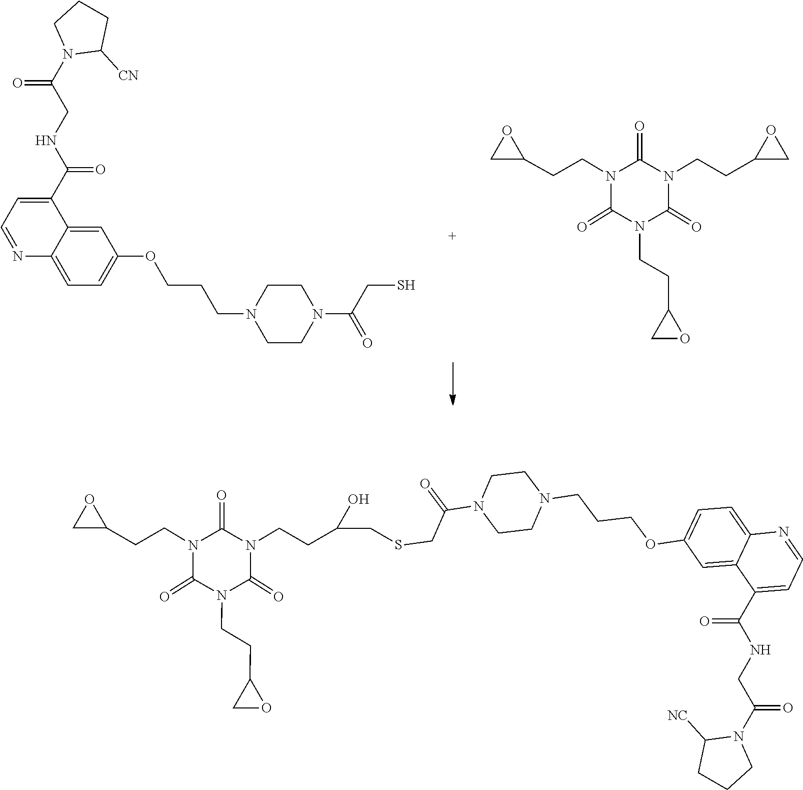 [SMILES: N#CC1CCCN1C(=O)CNC(=O)c1ccnc2ccc(OCCCN3CCN(C(=O)CS)CC3)cc12 ; O=c1n(CCC2CO2)c(=O)n(CCC2CO2)c(=O)n1CCC1CO1 ; N#CC1CCCN1C(=O)CNC(=O)c1ccnc2ccc(OCCCN3CCN(C(=O)CSCC(O)CCn4c(=O)n(CCC5CO5)c(=O)n(CCC5CO5)c4=O)CC3)cc12]

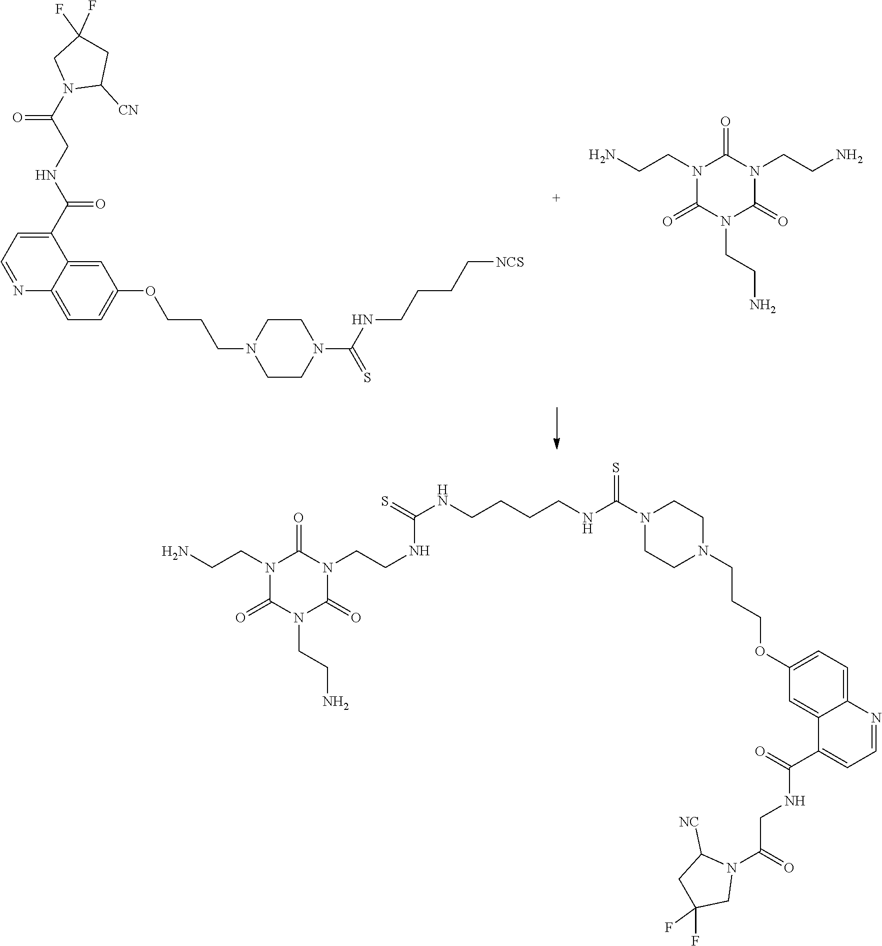

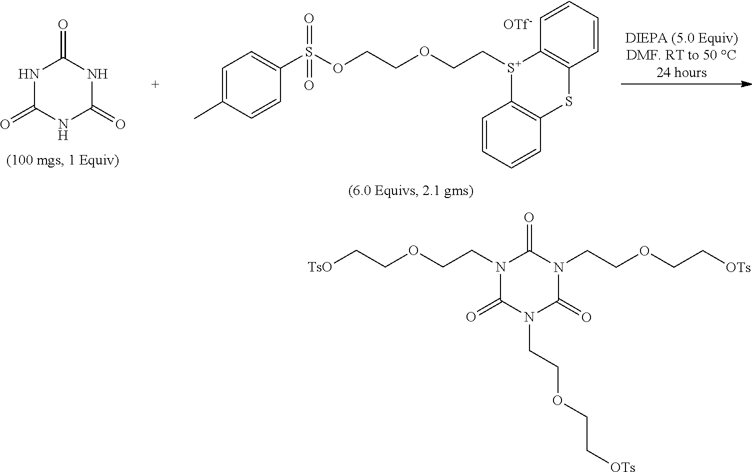 [SMILES: O=c1[nH]c(=O)[nH]c(=O)[nH]1 ; Cc1ccc(S(=O)(=O)OCCOCC[S+]2c3ccccc3Sc3ccccc32)cc1 ; Cc1ccc(S(=O)(=O)OCCOCCn2c(=O)n(CCOCCOS(=O)(=O)c3ccc(C)cc3)c(=O)n(CCOCCOS(=O)(=O)c3ccc(C)cc3)c2=O)cc1]

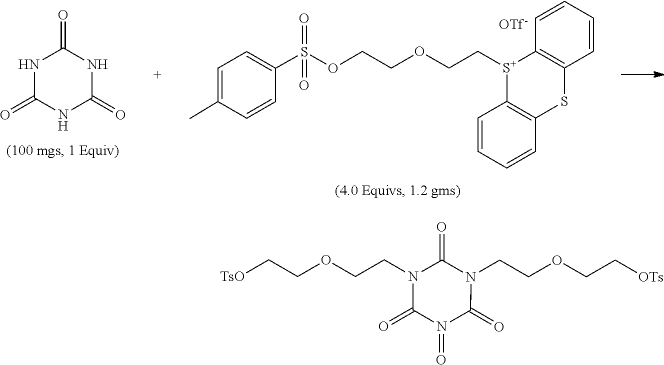

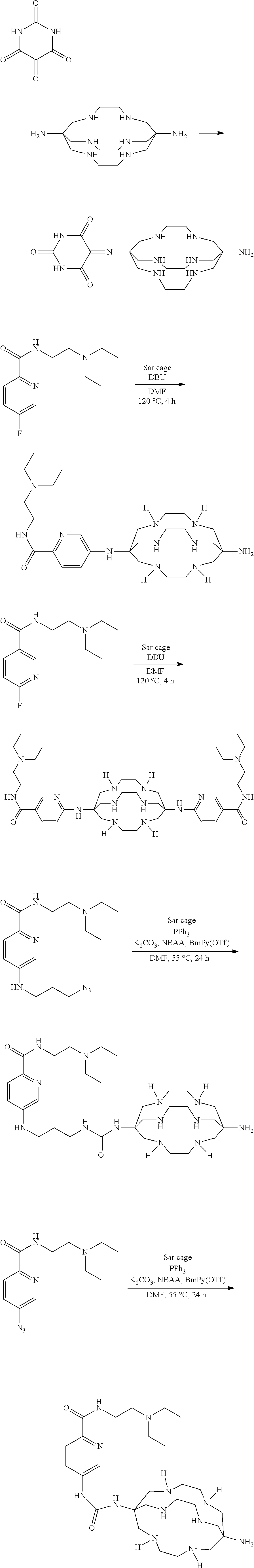

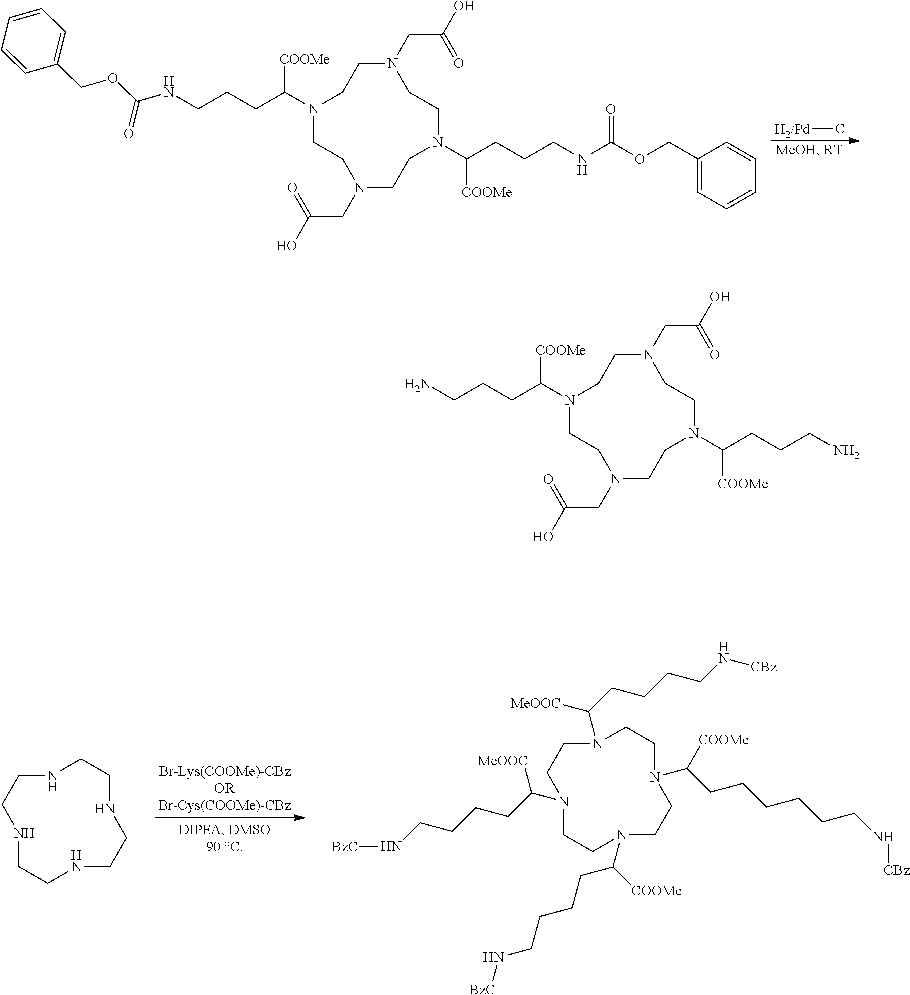

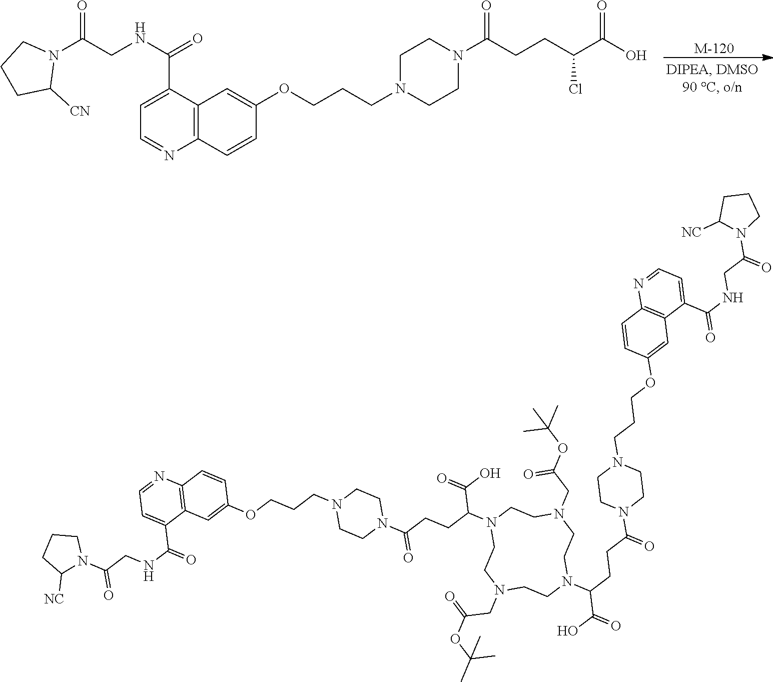

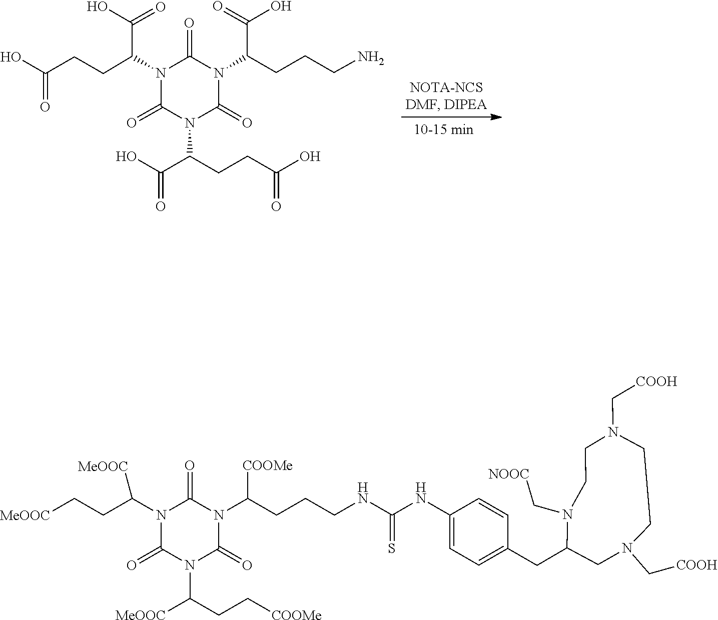

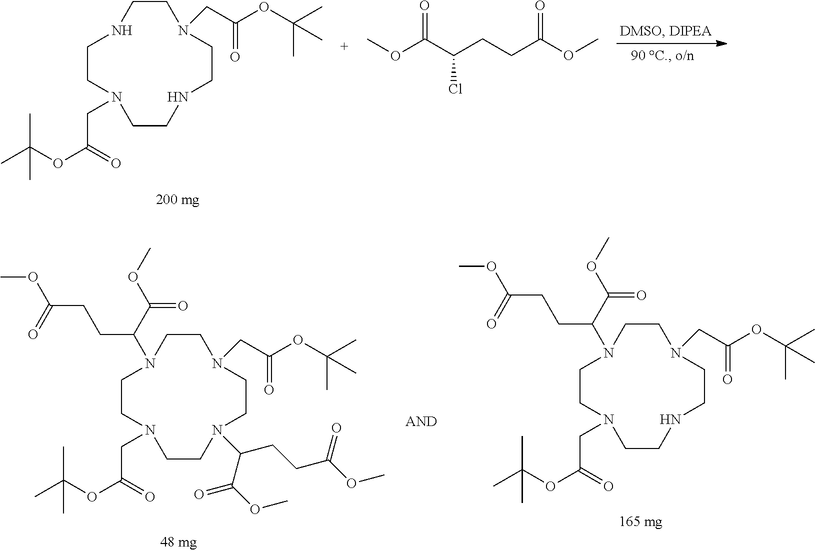

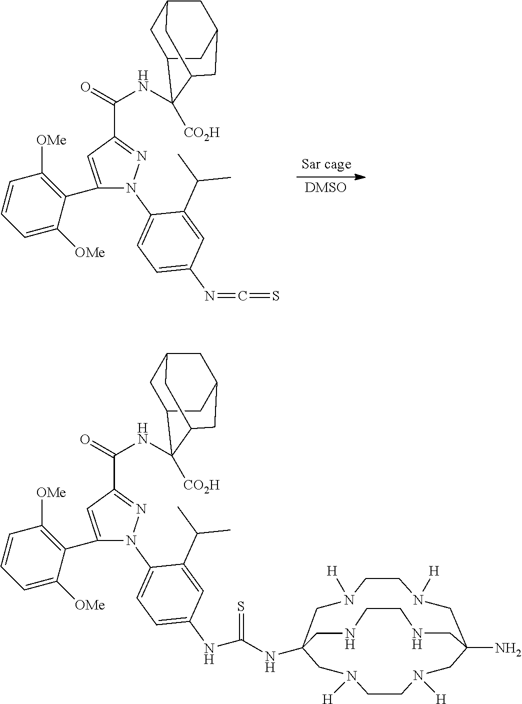 [SMILES: COc1cccc(OC)c1-c1cc(C(=O)NC2(C(=O)O)C3CC4CC(C3)CC2C4)nn1-c1ccc(N=C=S)cc1C(C)C ; COc1cccc(OC)c1-c1cc(C(=O)NC2(C(=O)O)C3CC4CC(C3)CC2C4)nn1-c1ccc(NC(=S)NC23CNCCNCC(N)(CNCCNC2)CNCCNC3)cc1C(C)C]

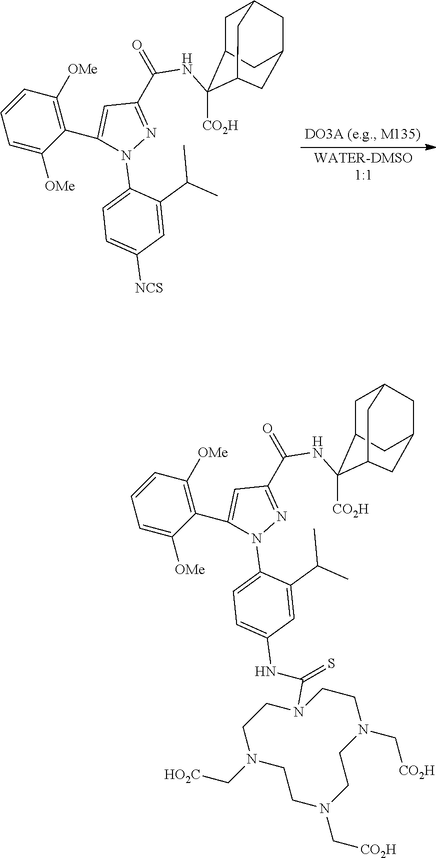

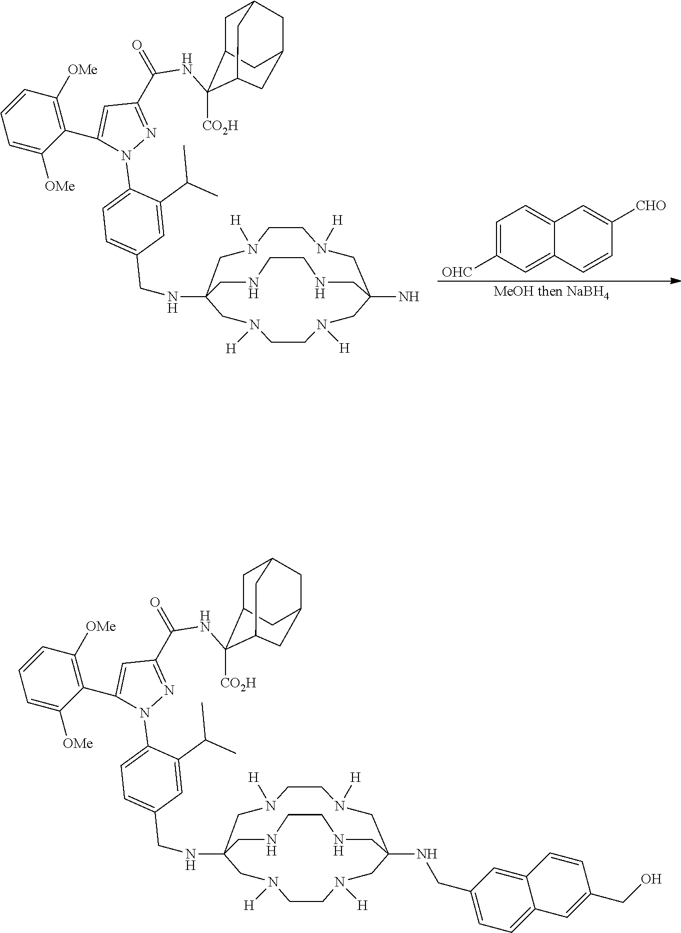 [SMILES: COc1cccc(OC)c1-c1cc(C(=O)NC2(C(=O)O)C3CC4CC(C3)CC2C4)nn1-c1ccc(CNC23CNCCNCC(N)(CNCCNC2)CNCCNC3)cc1C(C)C ; O=Cc1ccc2cc(C=O)ccc2c1 ; COc1cccc(OC)c1-c1cc(C(=O)NC2(C(=O)O)C3CC4CC(C3)CC2C4)nn1-c1ccc(CNC23CNCCNCC(NCc4ccc5cc(CO)ccc5c4)(CNCCNC2)CNCCNC3)cc1C(C)C]

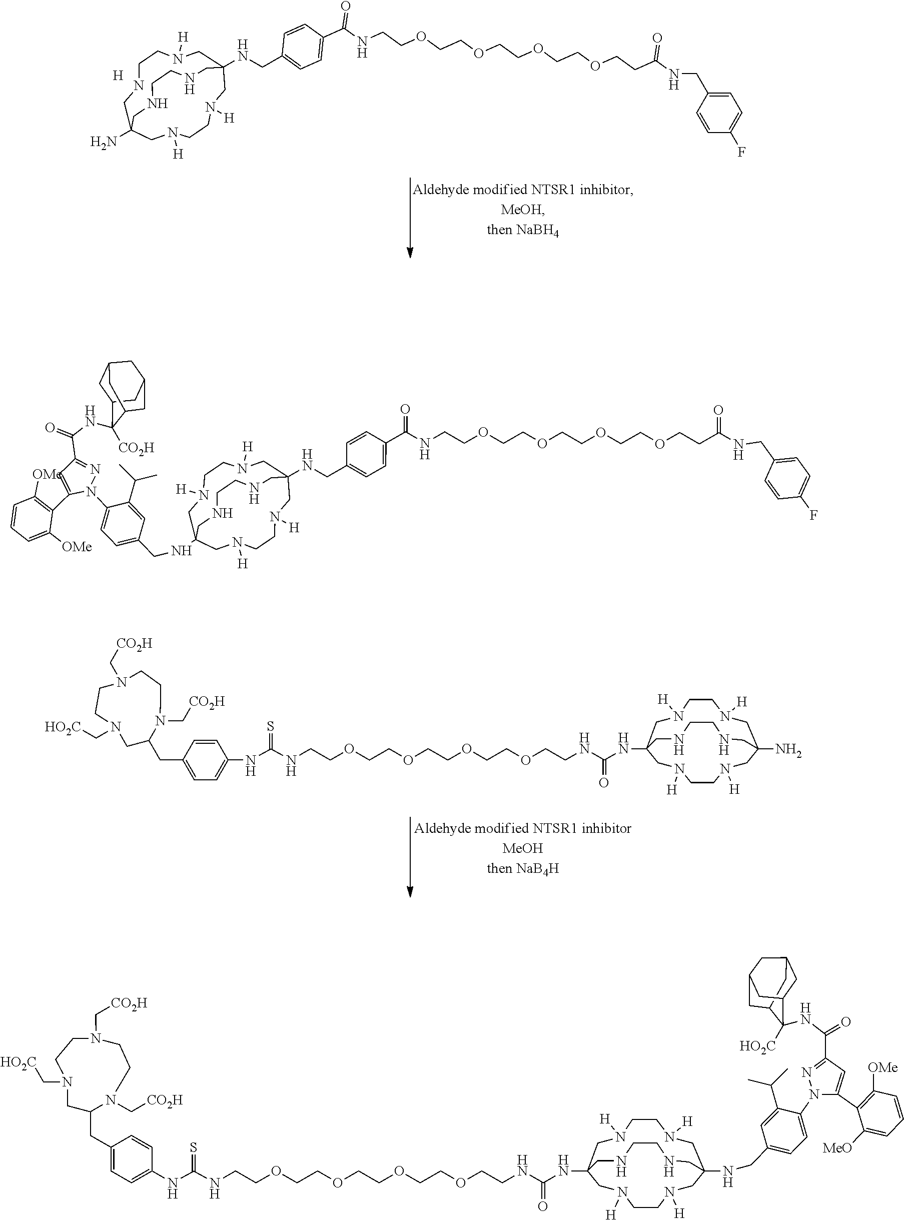

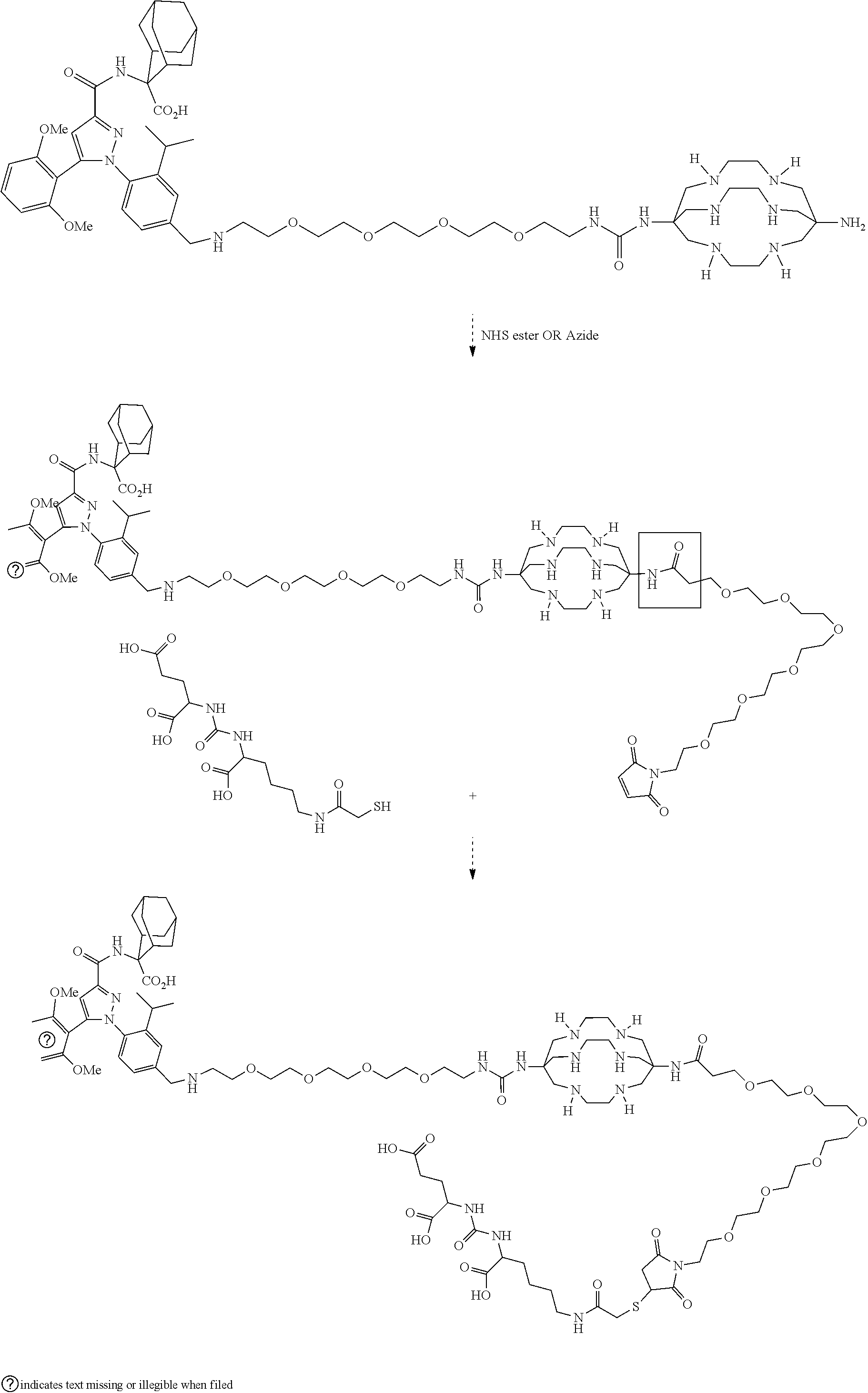

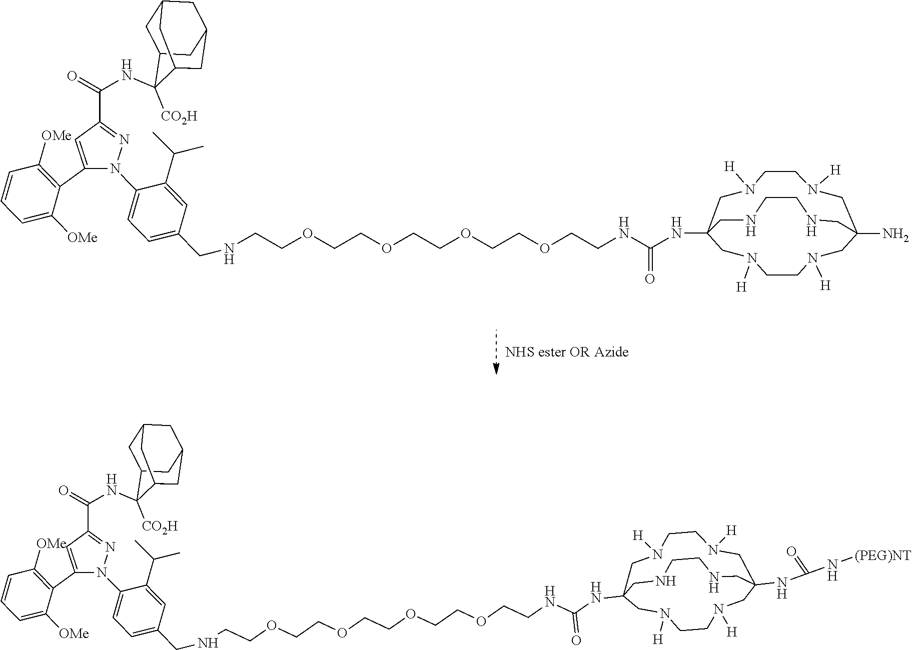

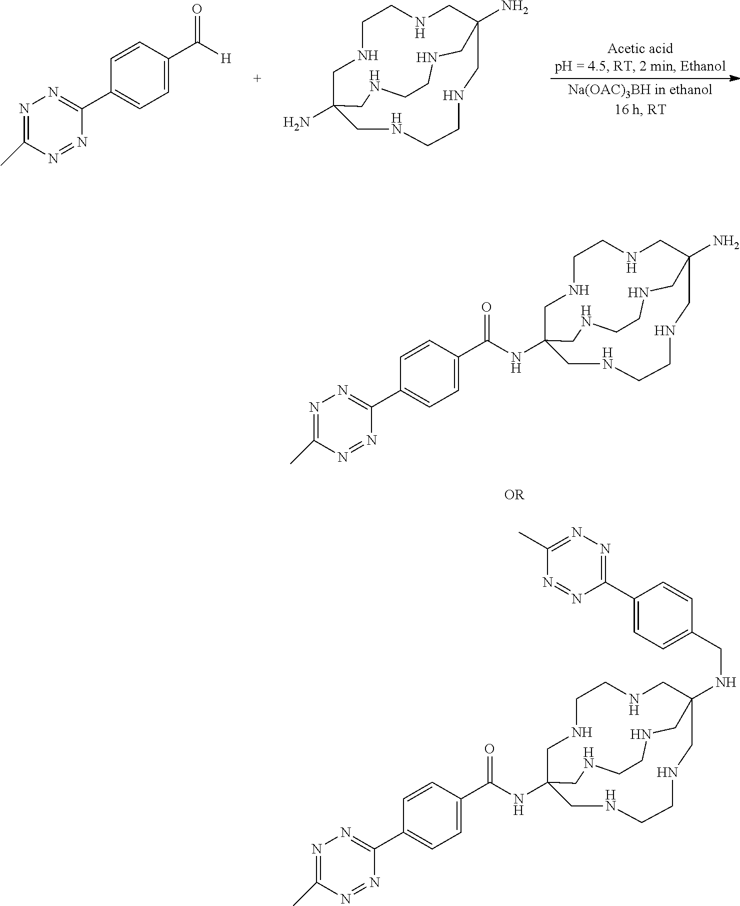 [SMILES: Cc1nnc(-c2ccc(C=O)cc2)nn1 ; NC12CNCCNCC(N)(CNCCNC1)CNCCNC2 ; Cc1nnc(-c2ccc(C(=O)NC34CNCCNCC(N)(CNCCNC3)CNCCNC4)cc2)nn1 ; Cc1nnc(-c2ccc(CNC34CNCCNCC(NC(=O)c5ccc(-c6nnc(C)nn6)cc5)(CNCCNC3)CNCCNC4)cc2)nn1]

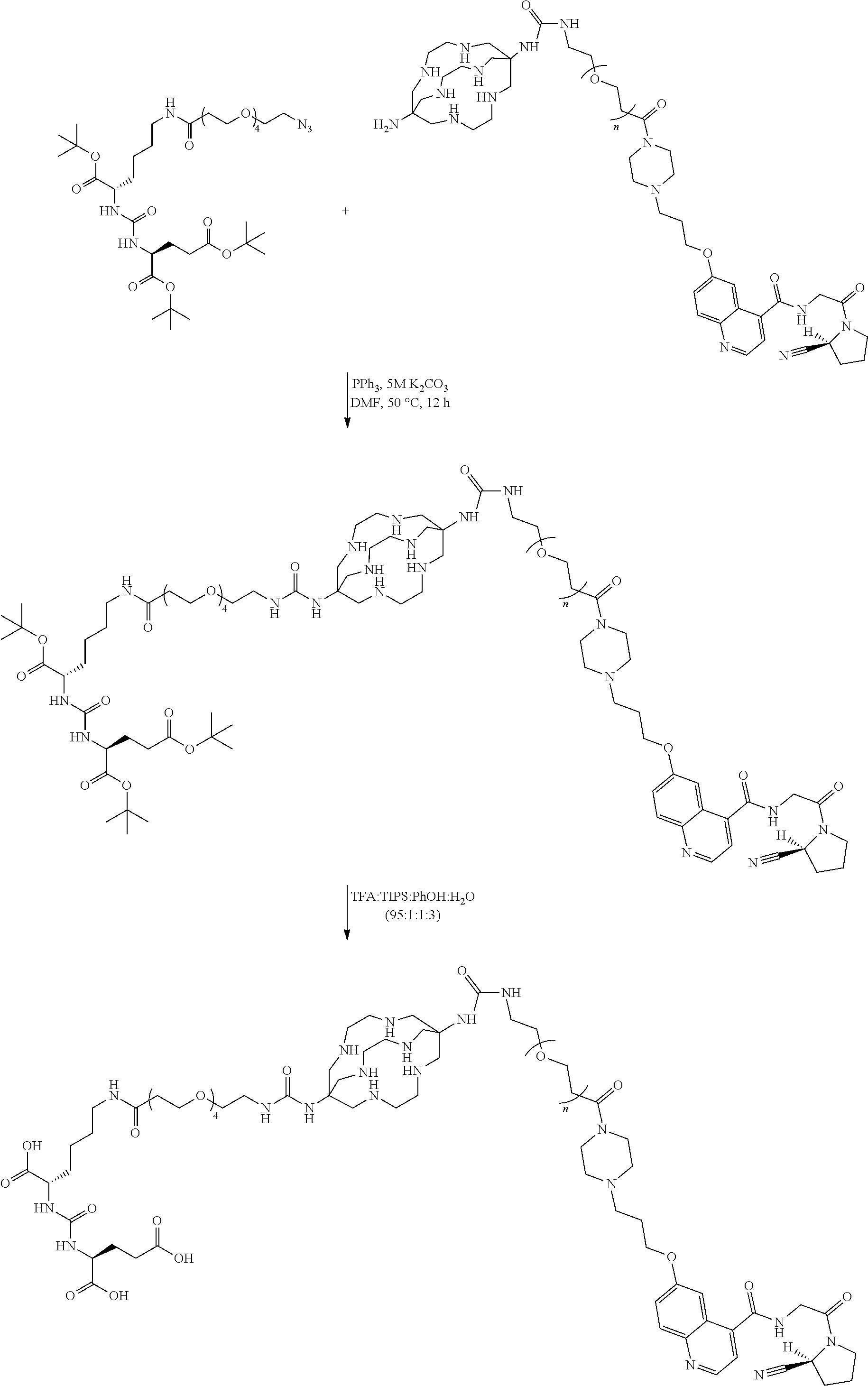

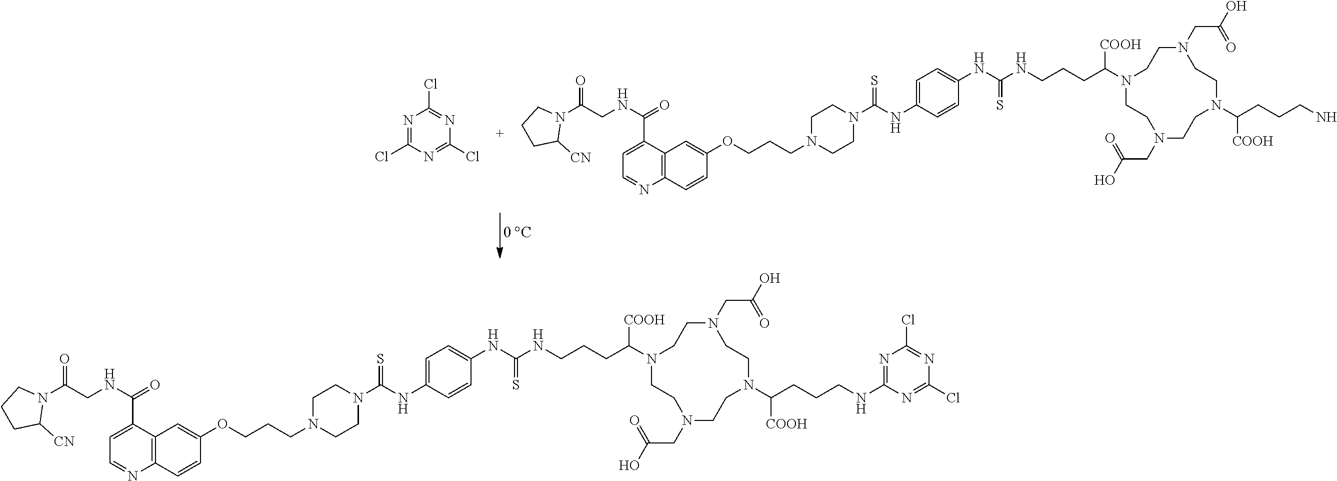 [SMILES: Clc1nc(Cl)nc(Cl)n1 ; N#CC1CCCN1C(=O)CNC(=O)c1ccnc2ccc(OCCCN3CCN(C(=S)Nc4ccc(NC(=S)NCCCC(C(=O)O)N5CCN(CC(=O)O)CCN(C(CCCN)C(=O)O)CCN(CC(=O)O)CC5)cc4)CC3)cc12 ; N#CC1CCCN1C(=O)CNC(=O)c1ccnc2ccc(OCCCN3CCN(C(=S)Nc4ccc(NC(=S)NCCCC(C(=O)O)N5CCN(CC(=O)O)CCN(C(CCCNc6nc(Cl)nc(Cl)n6)C(=O)O)CCN(CC(=O)O)CC5)cc4)CC3)cc12]

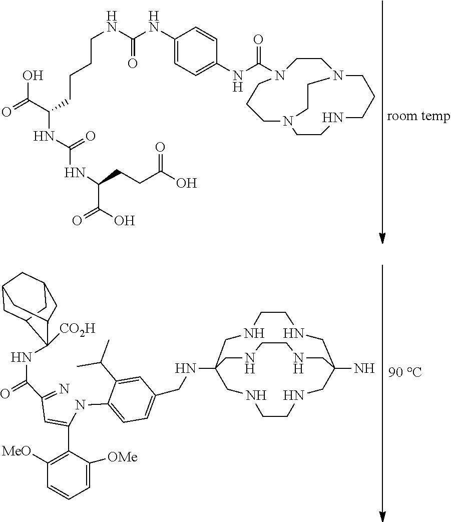 [SMILES: O=C(O)CC[C@H](NC(=O)N[C@@H](CCCCNC(=O)Nc1ccc(NC(=O)N2CCCN3CCNCCCN(CC3)CC2)cc1)C(=O)O)C(=O)O ; COc1cccc(OC)c1/C(=C/C(=N)C(=O)NC1(C(=O)O)C2CC3CC(C2)CC1C3)Nc1ccc(CNC23CNCCNCC(N)(CNCCNC2)CNCCNC3)cc1C(C)C]

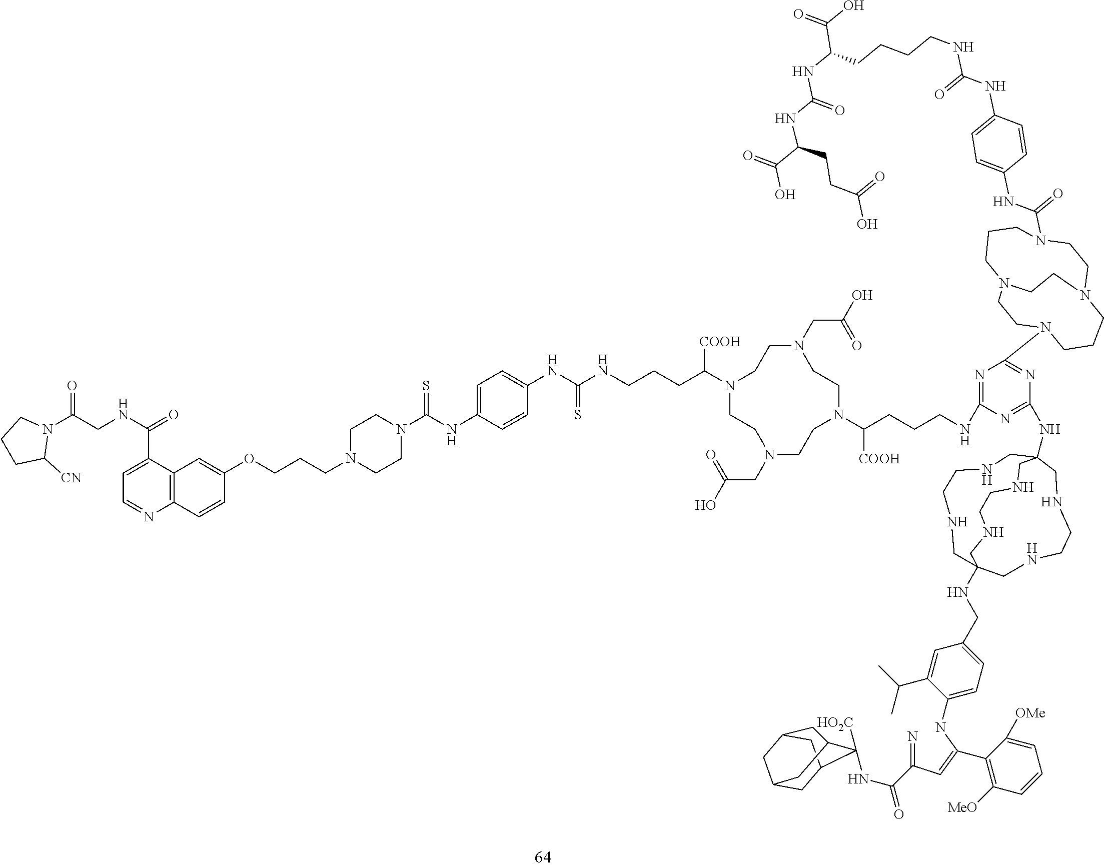 [SMILES: COc1cccc(OC)c1/C(=C/C(=N)C(=O)NC1(C(=O)O)C2CC3CC(C2)CC1C3)Nc1ccc(CNC23CNCCNCC(Nc4nc(NCCCC(C(=O)O)N5CCN(CC(=O)O)CCN(C(CCCNC(=S)Nc6ccc(NC(=S)N7CCN(CCCOc8ccc9nccc(C(=O)NCC(=O)N%10CCCC%10C#N)c9c8)CC7)cc6)C(=O)O)CCN(CC(=O)O)CC5)nc(N5CCCN6CCN(CCCN(C(=O)Nc7ccc(NC(=O)NCCCC[C@H](NC(=O)N[C@@H](CCC(=O)O)C(=O)O)C(=O)O)cc7)CC6)CC5)n4)(CNCCNC2)CNCCNC3)cc1C(C)C]

In some embodiments, the compound synthesis scheme is:

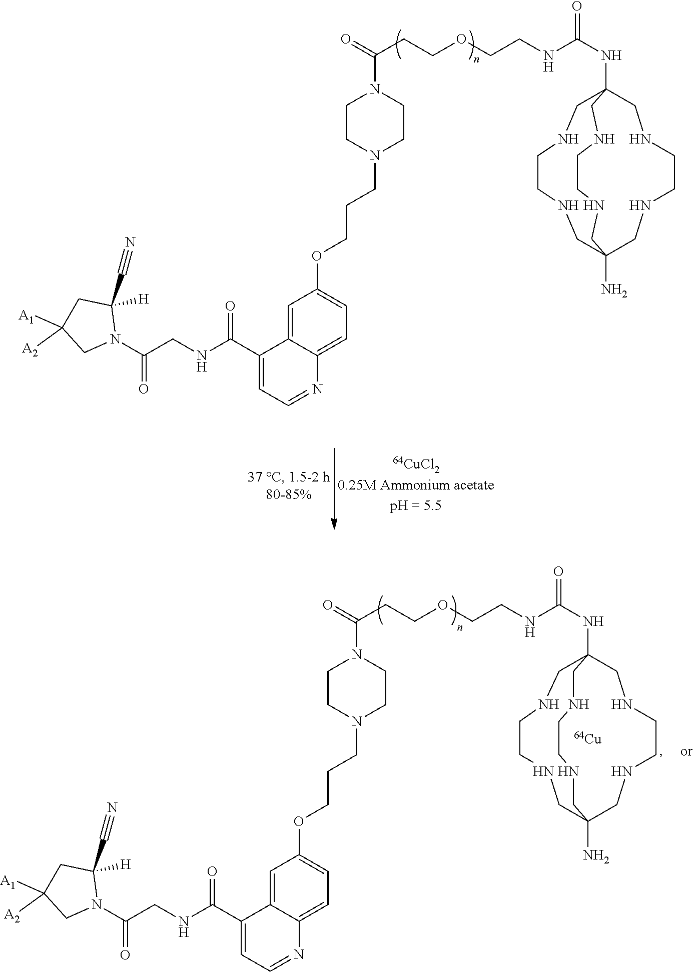

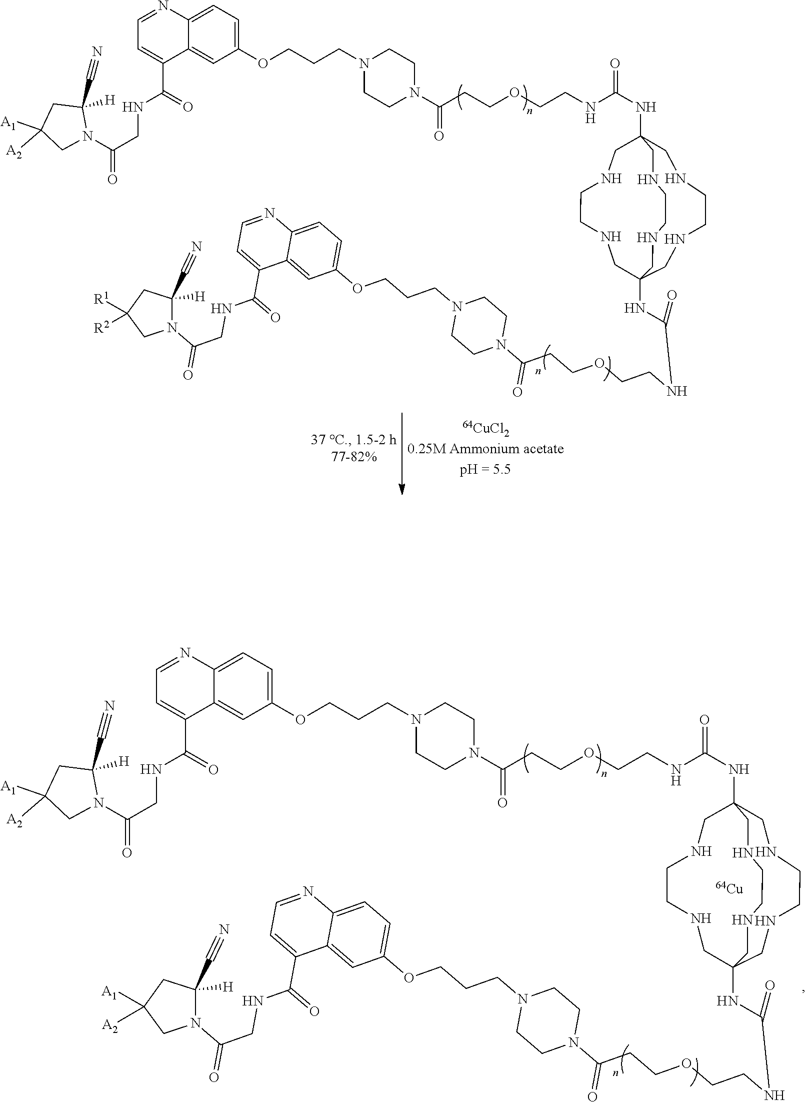

wherein A₁ and A₂ are each independently H or F, and n is an integer between 1 and 12; or a pharmaceutically acceptable salt thereof.
Other Example Compounds and their Uses.

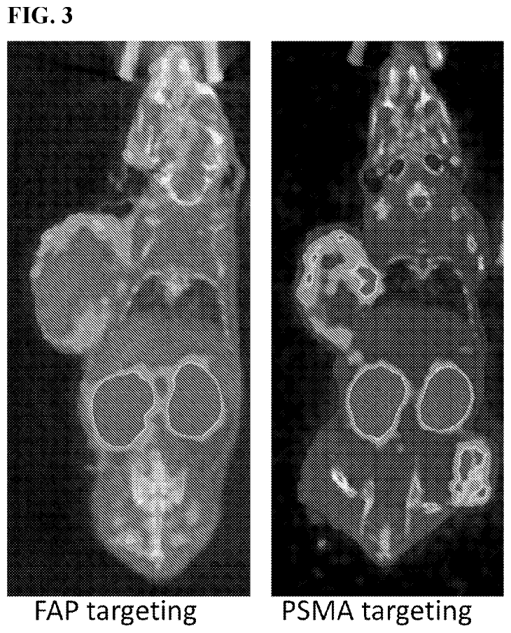

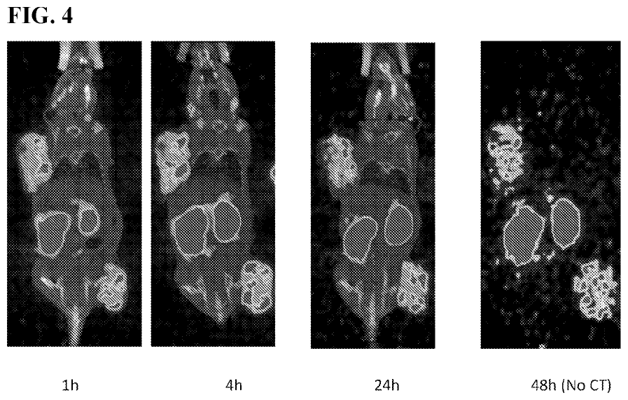

The modular nature of the compound cores, with the bi- and tri-functional attachments points, allows for compounds can (a) be constructed to target specific tumor types with different targeting ligands. (b) include open reactive groups that can bind to the target peptides and prolong time spent in proximity to the target cells, (c) include a chelator or be directly linked to a radionuclide for radiation therapy, (d) include chemotherapeutic groups for combination therapy with the aforementioned radiation therapy, and/or (e) include an optical/fluorescent dye or radionuclide (either through a chelator or covalent bond) for medical imaging. Utilization of different chemotherapeutics with a chelator and a PSMA/FAP targeting ligand are displayed below. The targeting ligands used in these compounds demonstrate uptake into tumors expressing either FAP or PSMA (FIG. 3). Including both a chelator, for therapeutic radionuclides, and a chemotherapeutic, such as a poly(ADP-ribose) polymerase (PARP) inhibitor, allows for one trifunctionalized molecule to be used as a single combination therapeutic (FIG. 4).

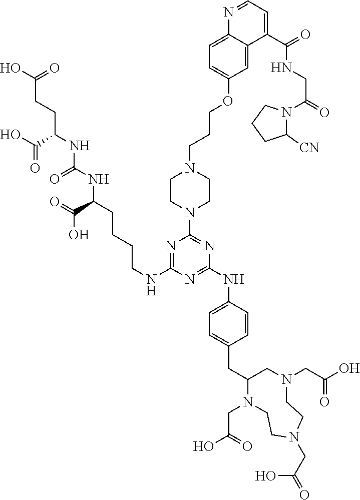 [SMILES: N#CC1CCCN1C(=O)CNC(=O)c1ccnc2ccc(OCCCN3CCN(c4nc(NCCCC[C@H](NC(=O)N[C@@H](CCC(=O)O)C(=O)O)C(=O)O)nc(Nc5ccc(CC6CN(CC(=O)O)CCN(CC(=O)O)CCN6CC(=O)O)cc5)n4)CC3)cc12]

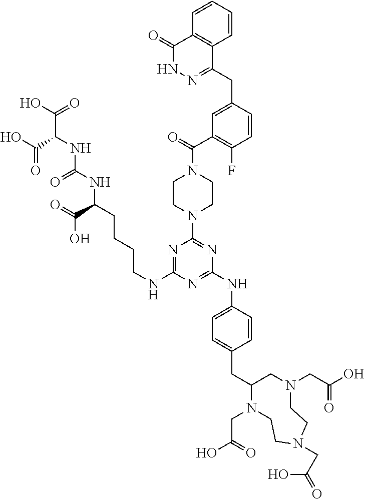

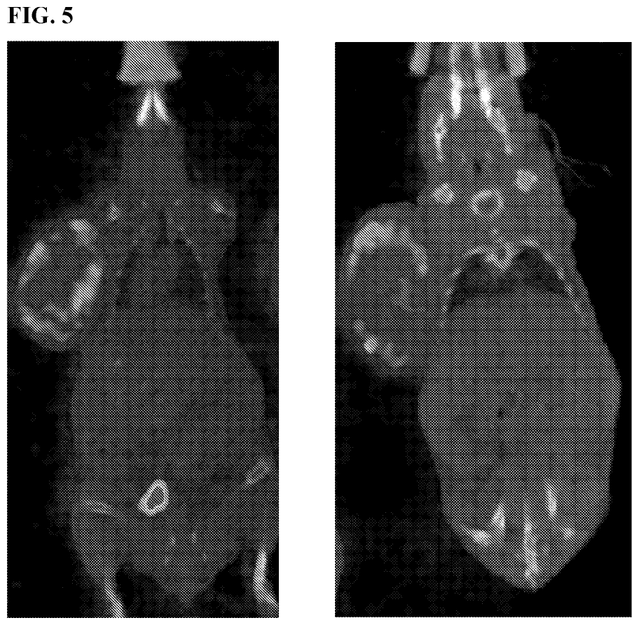

If only two functional groups are required, then a Sar cage may be used as the compound core. The Sar cage may also itself act as a chelator, adding to the flexibility of the platform. One example of this is shown below. Uptake of this compound into a tumor on a mouse is shown in FIG. 5.

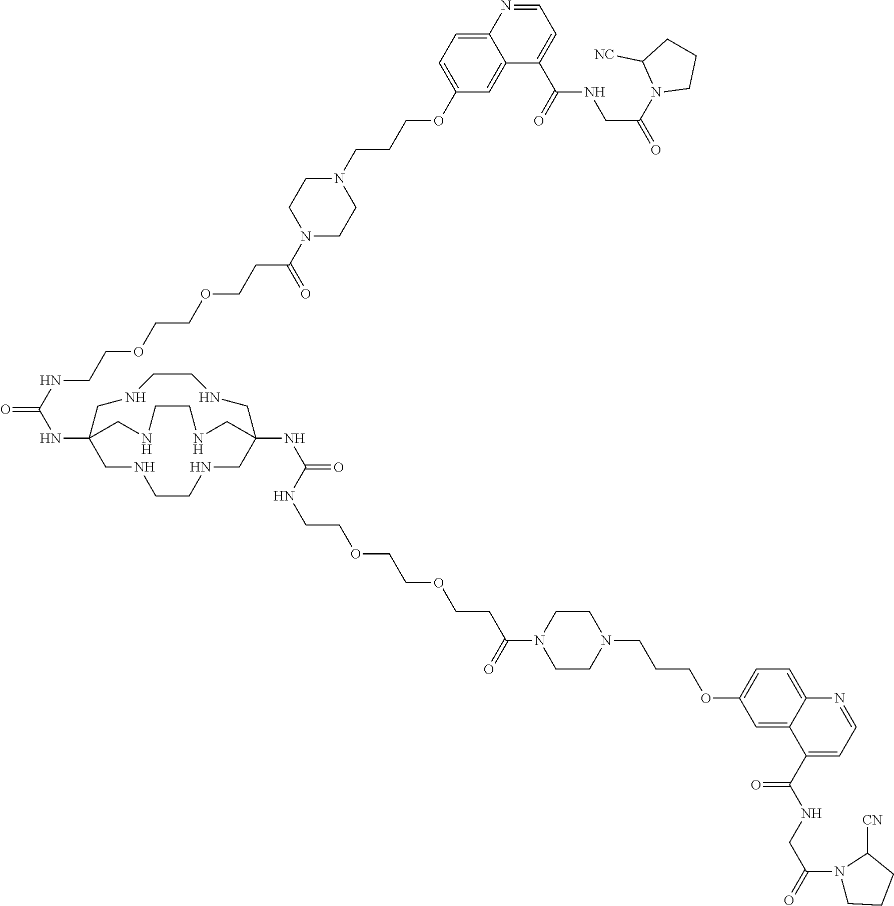 [SMILES: N#CC1CCCN1C(=O)CNC(=O)c1ccnc2ccc(OCCCN3CCN(C(=O)CCOCCOCCNC(=O)NC45CNCCNCC(NC(=O)NCCOCCOCCC(=O)N6CCN(CCCOc7ccc8nccc(C(=O)NCC(=O)N9CCCC9C#N)c8c7)CC6)(CNCCNC4)CNCCNC5)CC3)cc12]

On a trifunctional core, such as cyanuric acid, two of the functional groups may include a reactive sulfur atom which can then be used to form a cyclic peptide or a bicyclic peptide. Such cyclic peptides or bicyclic peptides can then be used as targeting probes, imaging agents, and therapeutics for the target tumors. Optionally, a chelating agent can be included in the peptide backbone of the cyclic peptide or bicyclic peptide. One such example compound is shown below.

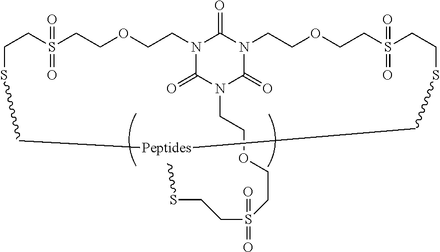

A compound with an open reactive group may improve retention when bound to the target cells. One such example is shown below.

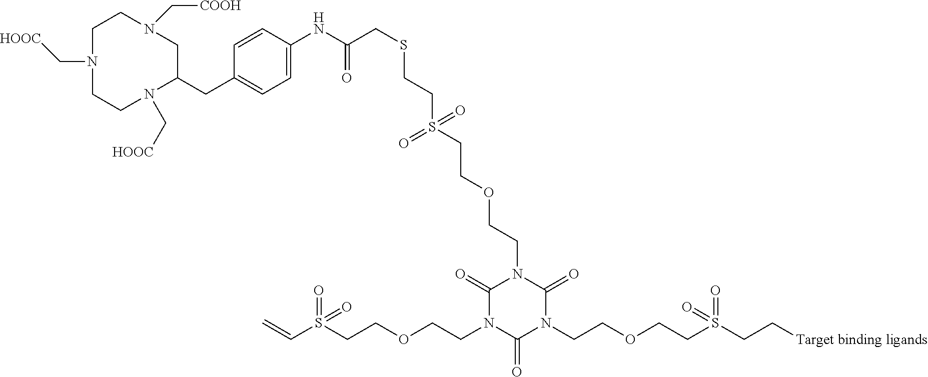

Certain radionuclides may be covalently linked to the core instead of using a chelator as an intermediary. One such example is shown below.

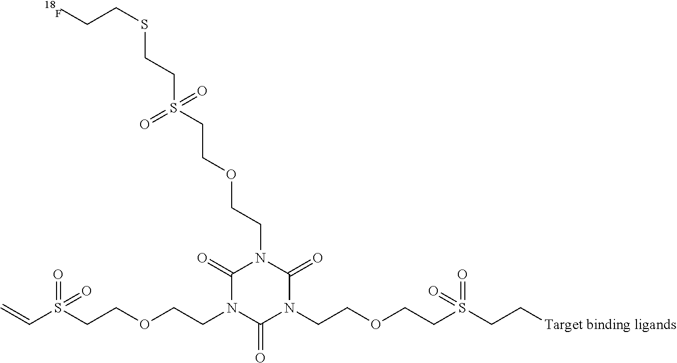

The Compound Core Impacts Compound Uptake into the Tumor.

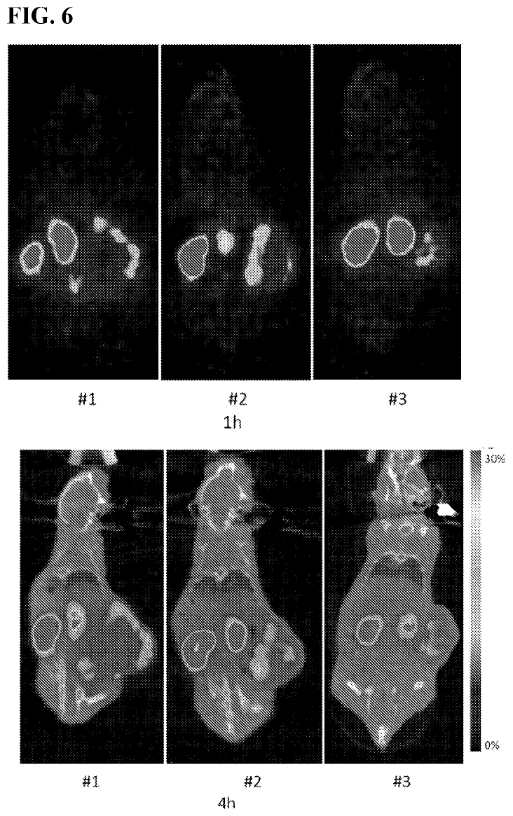

Attempts to use a simple PEG branching rather than a bifunctional or trifunctional compound core leads to poor uptake into the target tumor. An example of a compound constructed using PEG branching, rather than with a compound core of the current invention, is shown below. Uptake of this compound into a tumor is shown in FIG. 6.

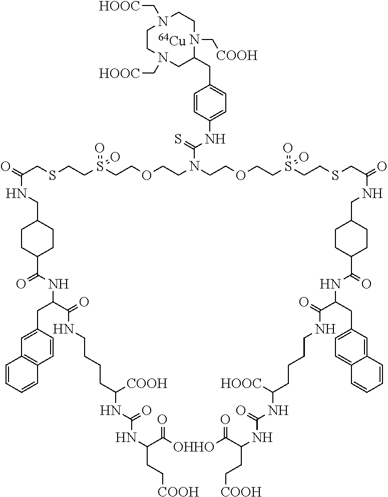

The foregoing is illustrative of the present invention, and is not to be construed as limiting thereof. The invention is defined by the following claims, with equivalents of the claims to be included therein.

1-104. (canceled)

105. A compound comprising a compound core which is a multifunctional heterocycle, wherein the compound core is covalently linked to at least one of a targeting ligand, a chelating agent, an imaging agent, a therapeutic agent, an open reactive group, a radionuclide, a cyclic peptide, or any combination thereof, or a pharmaceutically acceptable salt thereof, wherein the compound core is a bifunctional or trifunctional cyanuric acid, trithiocyanuric acid, triazine, barbituric acid, pyrimidine, Sar cage, 1,4,7,10-tetraazacyclododacane-1,4,7,10-tetraacetic acid (DOTA), 1,10-diaza-18-crown-6, or alloxan, or a homodimer, heterodimer, homotrimer, heterotrimer, homotetramer, or heterotetramer thereof.

106. The compound of claim 105, wherein the compound has the structure of Formula I: 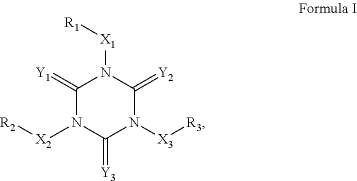
wherein:
R₁, R₂, and R₃ are independently absent or independently selected from the group consisting of the targeting ligand, the chelating agent, the imaging agent, the therapeutic agent, the open reactive group, the radionuclide, the cyclic peptide, and any combination thereof;
X₁, X₂, and X₃ are independently absent or a linker; and
Y₁, Y₂, and Y₃ are independently O or S;
wherein at least one of R₁, R₂, and R₃ is present.

107. The compound of claim 106, wherein R₁ is the targeting ligand, R₂ is the chelating agent, and R₃ is the imaging agent.

108. The compound of claim 106, wherein R₁ is the targeting ligand, R₂ is the chelating agent, and R₃ is the radionuclide.

109. The compound of claim 106, wherein R₁ is the targeting ligand, R₂ is the chelating agent, and R₃ is the therapeutic agent.

110. The compound of claim 106, wherein R₁ is the targeting ligand, R₂ is the imaging agent, and R₃ is the radionuclide.

111. The compound of claim 106, wherein R₁ is the targeting ligand, R₂ is the imaging agent, and R₃ is the therapeutic agent.

112. The compound of claim 106, wherein R₁ is the targeting ligand, R₂ is the open reactive group, and R₃ is the imaging agent.

113. The compound of claim 106, wherein R₁ is the targeting ligand, R₂ is the open reactive group, and R₃ is the therapeutic agent.

114. The compound of claim 106, wherein R₁ is the targeting ligand, R₂ is the open reactive group, and R₃ is the chelating agent.

115. The compound of claim 106, wherein R₁ is the targeting ligand, R₂ is the open reactive group, and R₃ is the radionuclide.

116. The compound of claim 106, wherein the targeting ligand is a neurotensin receptor (NTSR1) ligand, a prostate-specific membrane antigen (PSMA) ligand, a fibroblast activation protein (FAP) inhibitor, a C—X—C chemokine receptor type 4 (CXCR4) ligand, Bombesin (BBN), Arg-Gly-Asp (RGD), benzamide, folic acid and derivatives, a lipid and derivatives, melanin binding ligands (e.g., chloroquine, hydroxychloroquine, phenothiazines, and the like), choline and derivatives, an amino acid, a peptide, an antibody, glucose and derivatives, or other organic targeting molecules.

117. The compound of claim 106, wherein the imaging agent is for optical imaging, PET imaging, or SPECT imaging.

118. The compound of claim 106, wherein the chelating agent is selected from the group consisting of dimercaptopropanol, ethylenediaminotetraacetic acid (EDTA), diethylenetriaminepentaacetic acid (DTPA), octadentate macrocyclic bifunctional DOTA, DOTA-tert-butyl ester (M-120), 1,4,7,10-tetraazacyclododecane-1,4,7-triacetic acid trisodium salt (DO3A), hexadentate macrocyclic bifunctional 1,4,7-triazacyclononane-1,4,7-triacetic acid (NOTA), 2-S-(4-isothiocyanatobenzyl)-NOTA (NOTA-NCS), hydroxyethylidene diphosphonic acid (HEDP), ethylenediamine-N,N,N′,N′-tetrakis(methylenephosphonic acid) (EDTMP), 1,4,7,10-tetraazacyclododecane-1,4,7,10-tetraaminomethylenephosphonic acid (DOTMP), mercaptoacetyltriglycine (MAG₃), Sar cage, cross-bridged macrocyclic chelators (e.g., CB-Cyclam or CB-TE2A), salicylic acid, triethanolamine, ferrioxamine, macropa and derivatives, and ionophore.

119. The compound of claim 106, wherein the radionuclide is ¹¹C, ¹³N, ¹⁵O, ¹⁸F, ⁴⁴Sc, ⁵⁵Co, ^{58m}Co, ⁶¹Cu, ⁶⁴Cu, ⁶⁷Cu, ⁶⁷Ga, ⁶⁸Ga, ⁷⁶Br, ⁷⁷Br, ⁸²Br, ⁸⁹Zr, ⁸⁷Y, ⁹⁰Y, ^{99m}Tc, ¹¹¹In, ¹²³I, ¹²⁴I, ¹²⁵I, ¹³¹I, ¹³⁴Ce, ¹³⁴La, ¹⁴⁹Tb, ¹⁵²Tb, ¹⁵⁵Tb, ¹⁶¹Tb, ¹⁷⁷Lu, ¹⁸⁸Re, ²⁰¹Tl, ²¹¹At, ²⁰³Pb, ²¹²Pb, ²¹²Bi, ²¹³Bi, ²²³Ra, ²²⁵Ac, ²²⁶Th, ²²⁷Th, and/or ²³⁰U.

120. The compound of claim 106, wherein the therapeutic agent is a chemotherapeutic agent or an immunotherapeutic agent.

121. The compound of claim 106, wherein the targeting ligand, chelating agent, imaging agent, therapeutic agent, open reactive group, radionuclide, and/or cyclic peptide are linked to the compound core by a linker, wherein the linker comprises a polyethylene glycol (PEG), an alkyl, an alkoxy, an alkylsulfide, a sulfone, an amine, an alkylamino, an acyl, a carbonyl, a carboxylic acid, a methoxycarbonyl, a heterocycle, a heteroaryl, a polypeptide, and/or a vinyl sulfone.

122. The compound of claim 106, wherein the compound of Formula I is:

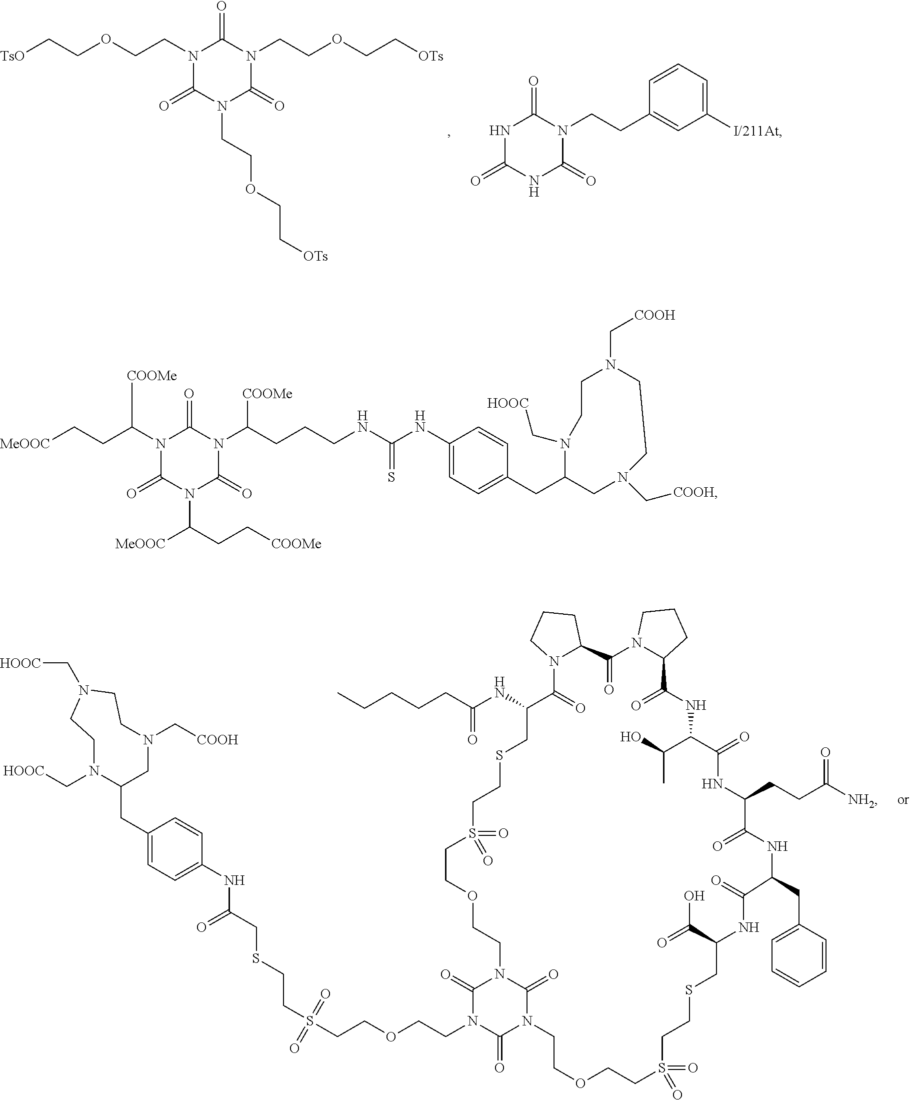

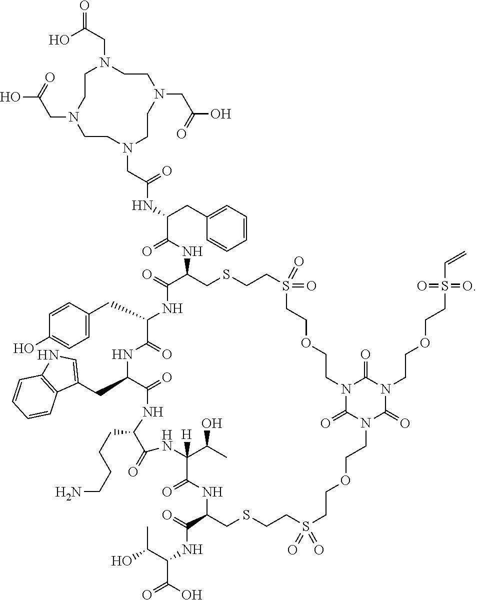 [SMILES: C=CS(=O)(=O)CCOCCn1c(=O)n2c(=O)n(c1=O)CCOCCS(=O)(=O)CCSC[C@@H](C(=O)N[C@H](C(=O)O)[C@@H](C)O)NC(=O)[C@@H]([C@H](C)O)NC(=O)[C@H](CCCCN)NC(=O)[C@@H](Cc1c[nH]c3ccccc13)NC(=O)[C@H](Cc1ccc(O)cc1)NC(=O)[C@@H](NC(=O)[C@@H](Cc1ccccc1)NC(=O)CN1CCN(CC(=O)O)CCN(CC(=O)O)CCN(CC(=O)O)CC1)CSCCS(=O)(=O)CCOCC2]

123. The compound of claim 106, wherein the compound of Formula I is a compound of Formula IA:

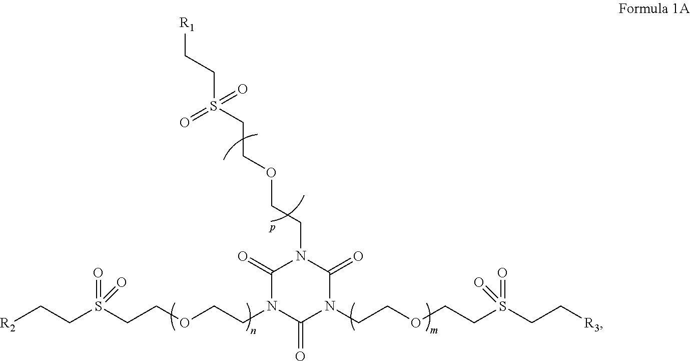

124. The compound of claim 123, wherein the compound of Formula IA is Compound 1: 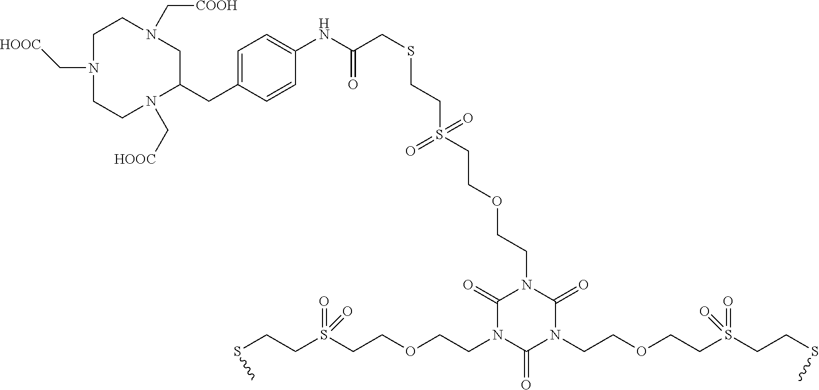
or a pharmaceutically acceptable salt thereof.

125. The compound of claim 123, wherein the compound of Formula IA is Compound 2: 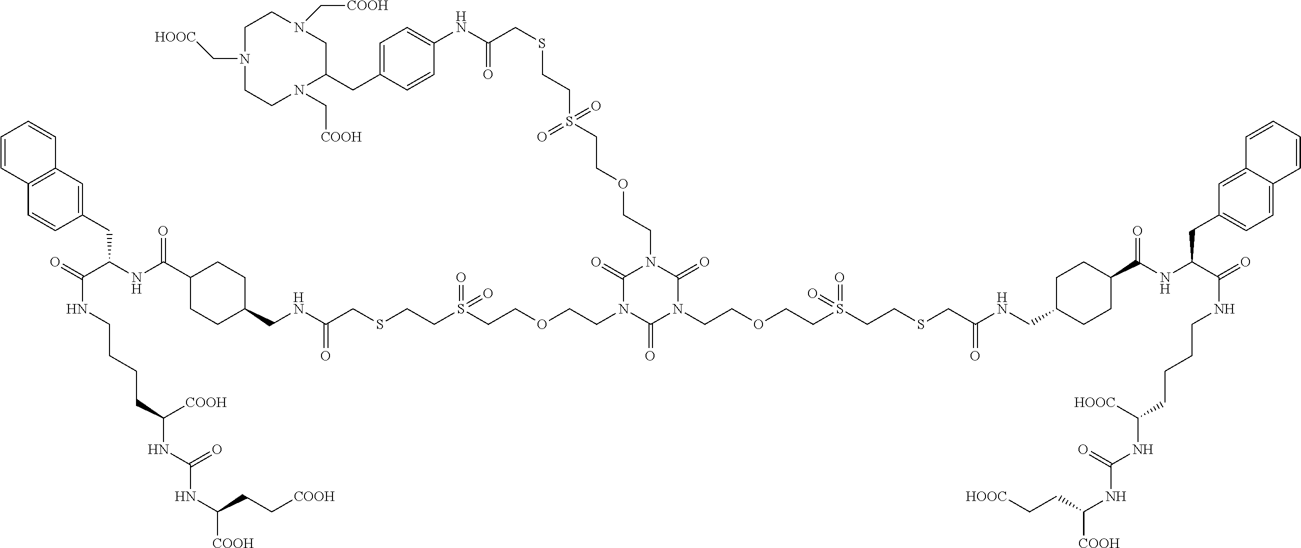
or a pharmaceutically acceptable salt thereof.
